(19) United States
(12) Patent Application Publication
(10) Pub. No.: US 2024/0424036 A1
(43) Pub. Date: Dec. 26, 2024

(54) COMPOSITIONS AND METHODS FOR PREVENTING OR TREATING INCONTINENCE, OVERACTIVE BLADDER, OR MENSTRUAL CRAMPING

(71) Applicants: Gregor Reid, London (CA); Jeremy P. Burton, London (CA); Behnam Abbasian, London (CA); Kimberly-Clark Worldwide, Inc., Neenah, WI (US)

(72) Inventors: Kathleen C. Engelbrecht, Kaukauna, WI (US); David W. Koenig, Menasha, WI (US); Elizabeth M. Doney, Appleton, WI (US); Gregor Reid, Arva (CA); Jeremy Burton, London (CA); Behnam Abbasian, London (CA)

(21) Appl. No.: 18/756,711

(22) Filed: Jun. 27, 2024

Related U.S. Application Data
(62) Division of application No. 17/415,020, filed on Jun. 17, 2021, now abandoned.
(60) Provisional application No. 62/783,465, filed on Dec. 21, 2018.

(51) Int. Cl. A61K 35/747; A61K 35/00

(52) U.S. Cl. CPC A61K 35/747; A61K 2035/115

(57) ABSTRACT

Methods and compositions for preventing or treating incontinence, overactive bladder, or menstrual cramping in a subject are described. A method can include providing a composition. The composition can include a modification agent. The modification agent can include a strain of Lactobacillus or a ferment or a metabolite thereof. The modification agent can provide neuromodulation activity by up-regulating at least one of maoA enzymes, maoB enzymes, and grin-1 genes. The method can also include administering the composition to the subject to prevent or treat incontinence, overactive bladder, or menstrual cramping. In some aspects, the modification agent can decrease a level of extracellular ATP in a target environment.

### BACKGROUND OF THE DISCLOSURE

Globally, about 800 million people have urinary incontinence (UI) and 70% are female. Urgency urinary incontinence (UUI) is one form of UI. Overactive bladder (OAB) can include muscle spasms of bladder muscles that can make a person feel as though they need to urinate, but may not leak urine. In some patients having UUI or OAB may experience the sensation of the need to instantly urinate regardless of whether the bladder is full. The sensing of a bladder being filled can involve various parts of a person's nervous system, and can eventually lead to contraction of bladder muscles, specifically the detrusor muscle, during micturition.

Despite the large number of people with UI, there is a lack of sufficient long term treatment. Various products exist to provide the ability to possibly lessen or manage incontinence symptoms without medical intervention, however, these products may involve insertion of various physical products or providing various stimulations near a person's bladder.

Additionally, menstrual cramping as part of a woman's menstrual period is a condition that a significant portion of women face on a regular basis. Menstrual cramping is due to the uterus contracting, and the amount and severity of cramping can vary based on various hormonal imbalances. Various treatment options exist to reduce menstrual cramping, however, opportunities for improvement and more natural solutions still exist.

Accordingly, a need exists for compositions and methods for preventing or treating incontinence, overactive bladder, or menstrual cramping in a subject.

### SUMMARY OF THE DISCLOSURE

It has now been surprisingly discovered that strains of Lactobacillus bacteria or ferments or metabolites thereof can serve as modification agents that can provide neuromodulation activity by up-regulating specific enzymes and genes that can lead to reduced contractility of muscles. Such modification agents can also reduce the amount of extracellular ATP in a target environment that can help reduce the contractility of muscles and organs, such as the bladder. Thus, the unexpected discovery of the modification agents of certain lactobacilli or ferments or metabolites thereof can be utilized in a composition to prevent or treat incontinence, overactive bladder, or menstrual cramping in a subject.

In one aspect, a method of preventing or treating incontinence, overactive bladder, or menstrual cramping in a subject is provided. The method can include providing a composition. The composition can include a modification agent comprising a strain of Lactobacillus or a ferment or a metabolite thereof. The modification agent can provide neuromodulation activity by up-regulating at least one of maoA enzymes, maoB enzymes, and grin-1 genes. The method can also include administering the composition to the subject to prevent or treat incontinence, overactive bladder, or menstrual cramping.

In another aspect, a method of preventing or treating incontinence, overactive bladder, or menstrual cramping in a subject is provided. The method can include providing a composition. The composition can include a modification agent comprising a strain of Lactobacillus or a ferment or a metabolite thereof. The modification agent can decrease an amount of ATP in a target environment. The method can also include administering the composition to the subject to prevent or treat incontinence, overactive bladder, or menstrual cramping.

In yet another aspect, a composition can include a carrier and a modification agent. The modification can include a strain of Lactobacillus gasseri or a ferment or a metabolite thereof selected from the group consisting of: Lactobacillus gasseri ATCC Designation No. PTA-125162, Lactobacillus gasseri ATCC Designation No. PTA-125163, and combinations thereof.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1A is a graph depicting fold change of maoA expression for a first set of samples.

FIG. 1B is a graph depicting fold change of maoA expression for a second set of samples.

FIG. 2A is a graph depicting fold change of maoB expression for the first set of samples.

FIG. 2B is a graph depicting fold change of maoB expression for the second set of samples.

FIG. 3A is a graph depicting fold change of grin-1 expression for the first set of samples.

FIG. 3B is a graph depicting fold change of grin-1 expression for the second set of samples.

FIG. 4 is a graph depicting an image intensity of the calcium influx of various samples from Lactobacillus or Lactococcus species.

FIG. 5A is a graph depicting muscle contraction versus time for samples of including Lactobacillus gasseri and/or Escherichia coli and DMEM.

FIG. 5B is a graph depicting muscle contraction versus time for samples of including Lactobacillus gasseri and/or Proteus mirabilis and DMEM.

FIG. 6A is a graph depicting the image intensity of calcium influx of various samples including LPS.

FIG. 6B is a graph depicting the image intensity of calcium influx of E. coli over time.

FIG. 6C is a graph depicting the image intensity of calcium influx of E. faecalis over time.

FIG. 7A is a graph depicting the image intensity of calcium influx of samples of E. coli and/or L. crispatus.

FIG. 7B is a graph depicting the image intensity of calcium influx of samples of E. coli and/or L. gasseri.

FIG. 7C is a graph depicting the image intensity of calcium influx of samples of serial dilutions of artificial urine (AU)/E. coli and L. crispatus/E. coli.

FIG. 8A is a graph depicting a quantification of extracellular ATP from E. coli and E. faecalis supernatants compared to AU.

FIG. 8B is a graph depicting a quantification of extracellular ATP from E. coli, L. crispatus, L. gasseri, and G. vaginalis from supernatants compared to AU.

FIG. 8C is a graph depicting a quantification of extracellular ATP from L. crispatus, L. gasseri, and L. vaginalis from supernatants compared to AU.

FIG. 8D is a graph depicting a quantification of extracellular ATP from L. crispatus grown in AU compared to AU supplemented with ATP.

FIG. 8E is a graph depicting a quantification of L. crispatus cultured in different concentrations of ATP.

FIG. 8F is a graph depicting a quantification E. coli cultured in different concentrations of ATP.

FIG. 8G is a graph depicting a quantification L. crispatus in AU supplemented with E. coli compared to L. crispatus in AU.

FIG. 8H is a graph depicting a quantification of L. crispatus in AU supplemented with G. vaginalis compared to L. crispatus in AU.

FIG. 8I is a graph depicting a quantification of extracellular ATP from various samples of E. coli and L. crispatus, and combination thereof, in AU.

FIG. 8J is a graph depicting a quantification of extracellular ATP from various samples of G. vaginalis and L. crispatus, and combination thereof, in AU.

FIG. 8K is a graph depicting the pH from samples of L. crispatus in AU compared to L. crispatus in a supernatant of G. vaginalis and AU.

FIG. 8L is a graph depicting a quantification of extracellular ATP from various samples of urothelial cells.

FIG. 9A is a graph depicting a quantification of E. coli cultured in various concentrations of ciprofloxacin (Cip).

FIG. 9B is a graph depicting a quantification of extracellular ATP from various samples of E. coli and ciprofloxacin.

FIG. 10A is a graph depicting the image intensity of calcium influx of samples of neurotransmitter γ-aminobutyric acid (GABA) and GABA included with ATP in comparison to ATP.

FIG. 10B is a graph depicting the image intensity of calcium influx of samples of GABA and GABA included with E. coli in comparison to E. coli.

FIG. 11A is a graph depicting the fold change for gene expression of maoA in E. coli and L. crispatus.

FIG. 11B is a graph depicting the fold change for gene expression of maoB in E. coli and L. crispatus.

FIG. 12A is a graph depicting the percent contraction of myofibroblast cells seeded on top of a collagen matrix tested against various bacterial samples including L. crispatus and/or E. coli.

FIG. 12B is a graph depicting the percent contraction of myofibroblast cells seeded on top of a collagen matrix tested against various bacterial samples including L. gasseri and/or E. coli.

FIG. 12C is a graph depicting the percent contraction of myofibroblast cells seeded on top of a collagen matrix tested against various samples including GABA.

FIG. 12D is a graph depicting the percent contraction of myofibroblast cells seeded on top of a collagen matrix tested against various bacterial samples including E. coli and E. coli with GABA.

FIG. 12E is a graph depicting the percent contraction of myofibroblast cells seeded on top of a collagen matrix tested against LPS.

FIG. 13A is a graph depicting the image intensity of α-SMA of samples of E. coli and L. crispatus compared to a sample of DMEM with FBS.

FIG. 13B is a graph depicting the fold change of alpha-smooth muscle actin (α-SMA) for L. crispatus and/or E. coli compared to a sample of DMEM with FBS.

FIG. 13C is a graph depicting the fold change of TNF-α for L. crispatus and/or E. coli compared to a sample of DMEM with FBS.

### DEFINITIONS

As used herein, the term “absorbent article” refers to an article which may be placed against or in proximity to the body (i.e., contiguous with the body) of the wearer to absorb and contain various liquid, solid, and semi-solid exudates discharged from the body. It is to be understood that the present disclosure is applicable to various disposable absorbent articles, including, but not limited to, diapers, diaper pants, training pants, youth pants, swim pants, feminine hygiene products, including, but not limited to, menstrual pads or pants, incontinence products, medical garments, surgical pads and bandages, other personal care or health care garments, and the like without departing from the scope of the present disclosure.

As used herein, the term “ferment” and “metabolites” refers to any output, by-product, or substance produced as a result of culturing bacteria, including a supernatant, plus any remaining bacteria (dead or alive). A ferment or metabolite may be further processed by extraction, filtration, and/or other procedures.

### DETAILED DESCRIPTION OF THE DISCLOSURE

The present invention is related to compositions and methods useful in preventing or treating incontinence, overactive bladder, or menstrual cramping in a subject. The compositions can be configured to be administered to a subject through topical application in various forms, including, but not limited to, a liquid, cream, gel, or spray. Compositions can be alternatively or additionally administered to a subject through a delivery mechanism such as, for example, a wipe substrate or by being applied to an absorbent article that can deliver the composition to the subject. Another way the compositions may be configured to be administered to a subject can be by having the composition be configured in the form of a pill that can be ingested by the subject.

Although it may be difficult to envision, it is believed that the detrusor muscle which controls micturition could be affected by bacteria present at the urothelial layer. The urothelium is only 3-5 mm thick, and uropathogens are known to damage and invade this layer. The urothelial cells communicate with the sub-urethral tissue which contains nervous system and myofibroblast, and with smooth muscle cells, by releasing some excitatory compounds. It is believed that bacterial compounds could induce urothelial cells to further release excitatory compounds into the sub-urethral space and send signals to the brain falsely indicating that the lumen is under tension associated with a full bladder, thereby inducing smooth muscle contraction and voiding leading to UUI or OAB. It is believed that certain commensal bacteria play a role in interfering with UUI pathogenesis, in a manner that might allow better control of contractions of the bladder muscles.

A variety of bacterial strains were collected, identified, and tested for neuromodulation activity. The neuromodulation activity was tested through looking for an increase in fold change of monoamine oxidase A and B enzymes (maoA and maoB) and an increase in grin-1 genes. These specific enzymes and gene were selected for testing because maoA and maoB are enzymes that degrade biological amines, including the neurotransmitters serotonin, norepinephrine, phenylethylamines, and dopamine. These neurotransmitters are known to play a role in the frequency of urination, the maximum urine capacity of the bladder, and the contraction of bladder muscles. Grin-1 is a critical subunit of a glutamate receptor that forms a gated ion channel. The ion channel controls the flow of Ca2+, a key divalent cation that is required for bladder muscle contraction.

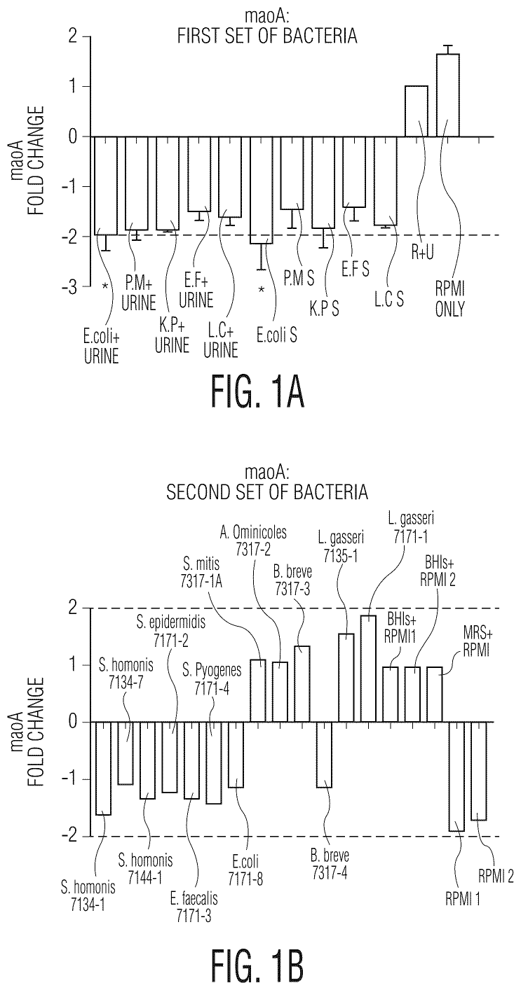

For the neuromodulation activity testing, two sample sets of bacteria were tested. A first sample set of bacteria that will be described for testing results of FIGS. 1A, 2A, and 3A included Escherichia coli, Proteus mirabilis, Klebsiella pneumoniae, Enterococcus faecalis, Lactobacillus crispatus. These bacterial samples were tested in artificial urine, as well as samples in supernatant (referenced with an S designation in FIGS. 1A, 2A, and 3A). Artificial urine used in this testing is described in the Materials and Test Methods section herein. The bacterial samples were Roswell Park Memorial Institute medium (RPMI) was also tested as a control, both in urine as well as by itself.

The second set of bacteria was collected from urine from various subjects in a study. Included in the study were subjects that suffered from UUI, as well as subjects that did not suffer from UUI. The urine was collected via catheter, and therefore, the bacteria collected in the second set of bacteria for testing is from the bladder and not from the urethra or outer urogenital sites. The urine was cultured for viable bacteria using expanded quantitative culture technique, as described by the journal article of Hill et al. (Hilt, E. E., Mckinley, K., Pearce, M. M., Rosenfeld, A. B., Zilliox, M. J., Mueller, E. R., Brubaker, L., Gai, X., Wolfe, A. J., and Schreckenberger, P. C. (2013). Urine Is Not Sterile: Use of Enhanced Urine Culture Techniques To Detect Resident Bacterial Flora in the Adult Female Bladder. Journal of Clinical Microbiology, 52(3), 871-876. Retrieved Mar. 1, 2016). The second set of bacteria will be described for the testing results of FIGS. 1B, 2B, and 3B and included Staphylococcus hominis 7134-1, Staphylococcus hominis 7134-7, Staphylococcus hominis 7144-1, Staphylococcus epidermidis 7171-2, Enterococcus faecalis 7171-3, Streptococcus pyogenes 7171-4, Escherichia coli 7171-8, Streptococcus mitis 7317-1A, Alloscardovia omnicolens 7317-2, Bifidobacterium breve 7317-3, Bifidobacterium breve 7317-4, Lactobacillus gasseri 7135-1, and Lactobacillus gasseri 7171-1. Also tested in the second set of bacteria testing was Brain Heart Infusion medium (BHIs) and RPMI 1, BHIs and RPMI 2, De Man, Rogosa, and Sharpe agar (MRS) and RPMI, RPMI 1, and RPMI 2 tested as controls. The bacteria from the second set was identified via matrix-assisted laser desorption/ionization time of flight mass spectrometry (MALDI-TOF). The second set of bacteria were incubated with human bladder cell lines (5637 and T24).

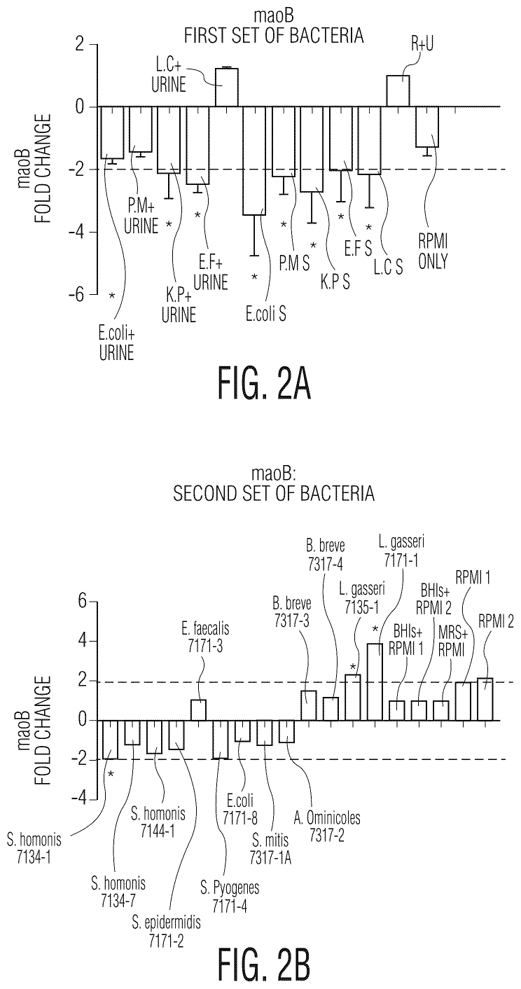

Turning now to FIGS. 1A and 1B, a gene expression study was conducted to determine the fold change of the expression of maoA for the first set of bacteria samples (FIG. 1A) and for the second set of bacteria samples (FIG. 1B). As illustrated in FIGS. 1A and 1B, the Lactobacillus gasseri strains in FIG. 1B demonstrated a significant increase in fold change in the maoA compared to other bladder bacteria strains. FIGS. 2A and 2B tested the first and second set of bacteria samples, respectively, for gene expression of maoB, and similar to FIGS. 1A and 1B, the L. gasseri strains demonstrated an up-regulation of maoB compared to other bacterial strains tested.

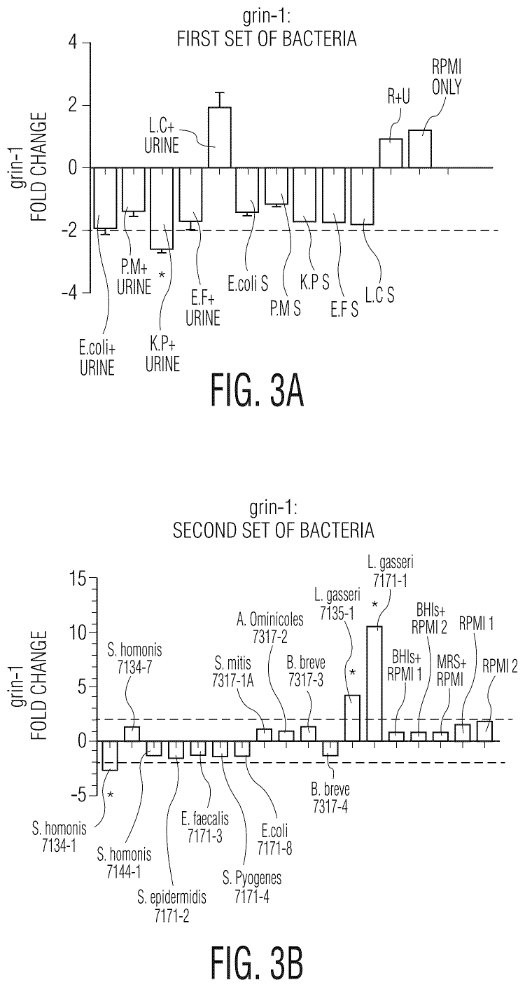

FIGS. 3A and 3B depict the fold change of expression of grin-1 genes for the first and second set of bacterial samples, respectively. The L. gasseri bacterial strains of the second set of bacterial samples shown in FIG. 3B again demonstrated a significant up-regulation of grin-1 expression compared to the other bacterial samples. The asterisks used in FIGS. 1A-3B demonstrate a significant increase or difference.

Reviewing the enzyme and gene expression testing results of FIGS. 1A-3B, it is worth noting that the bacteria samples including Lactobacillus crispatus also provided some up-regulation of maoB (FIG. 2A) and grin-1 (FIG. 3A), however, not to the same level of up-regulation of maoB and grin-1 as the Lactobacillus gasseri strain. Additionally, the Lactobacillus crispatus bacterial samples did not up-regulate the maoA enzyme as depicted in FIG. 1A. This provided a surprising result that the Lactobacillus gasseri bacterial strains tested provided up-regulation of all three maoA, maoB, and grin-1, while the bacteria of Lactobacillus crispatus from the same genus only provided a slight up-regulation of maoB and grin-1, and down-regulated the maoA enzyme.

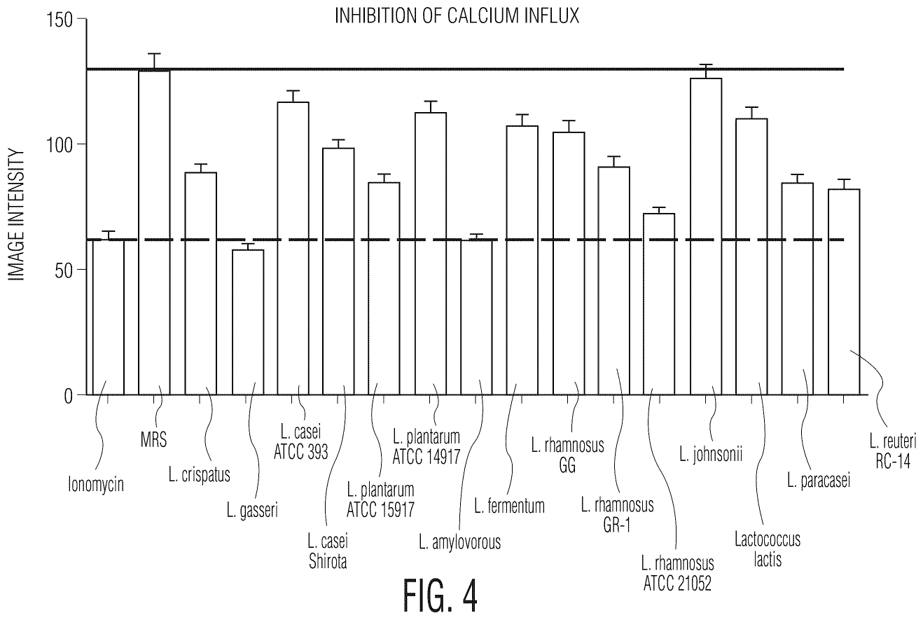

Further testing was conducted to determine the inhibition of calcium influx of a variety of bacteria samples. The bacterial samples tested in this testing was lonomycin, MRS, Lactobacillus crispatus, Lactobacillus gasseri KE-1 (ATCC Designation No. PTA-125162), Lactobacillus casei ATCC 393, Lactobacillus casei Shirota, Lactobacillus plantarum ATCC 15917, Lactobacillus plantarum ATCC 14917, Lactobacillus amylovorous, Lactobacillus fermentum, Lactobacillus rhamnosus GG, Lactobacillus rhamnosus GR-1, Lactobacillus rhamnosus ATCC 21052, Lactobacillus johnsonii, Lactococcus lactis, Lactobacillus paracasei, and Lactobacillus reuteri RC-14. The results of the inhibition of calcium influx are illustrated in FIG. 4. The dashed line in FIG. 4 indicates an inhibition of the influx of calcium, whereas the solid line indicates a stimulated influx of calcium. As depicted in FIG. 4, despite increasing the amount expression of grin-1, Lactobacillus gasseri did not stimulate the influx of calcium in the urothelium. In fact, Lactobacillus gasseri inhibited the influx of calcium. In comparison, many other bacterial species of the Lactobacillus genus that were tested surprisingly had much higher levels of calcium intensity levels, or a lower inhibition against calcium influx. As previously discussed, Ca2+ ions are a key divalent cation that are required for bladder muscle contraction, and thus, the ability of Lactobacillus gasseri to inhibit calcium influx is believed that the bacteria, or ferments thereof, may help reduce the contractions associated with UUI and OAB.

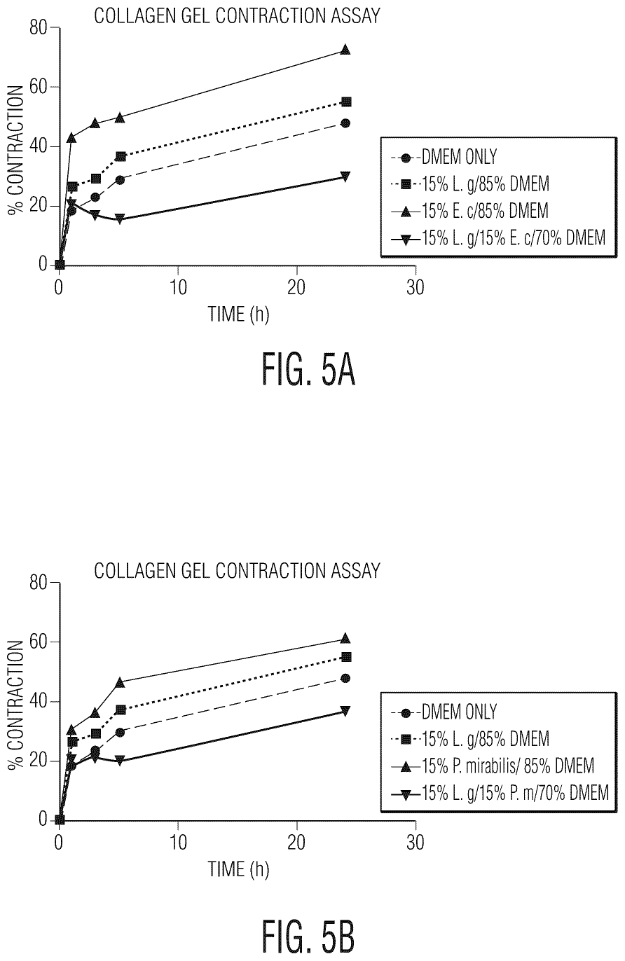

Turning to FIGS. 5A and 5B, Lactobacillus gasseri supernatant was demonstrated to slow contractions in a collagen gel contraction assay compared to supernatants from uropathogens Escherichia coli and Proteus mirabilis. Additionally, contractions were also slowed when L. gasseri supernatant was tested in combination with supernatants from E. coli and P. mirabilis.

The specific Lactobacillus gasseri strains tested collected, cultured, and tested herein were deposited with the ATCC. Lactobacillus gasseri 7135-1 discussed above received American Type Culture Collection (ATCC) Designation No. PTA-125163 (strain DK1). Genomic sequencing of Lactobacillus gasseri 7135-1 (ATCC Designation No. PTA-125163) was completed and was published, receiving Accession Numbers with the National Center for Biotechnology Information (NCBI) of BioSample: SAMN11332831 (Sample Name: LGASSERI-7135) and BioProject: PRJNA530717. Lactobacillus gasseri 7171-1 discussed above received ATCC Designation No. PTA-125162 (strain KE1). Genomic sequencing of Lactobacillus gasseri 7171-1 (ATCC Designation No. PTA-125162) was completed and was published, receiving Accession Numbers with the NCBI of BioSample: SAMN11332833 (Sample Name: LGASSERI-7171) and BioProject: PRJNA530718. Optimally, the L. gasseri strains are grown on MRS agar or broth under anerobic conditions.

Additional testing was completed on the role of extracellular release of adenosine triphosphate (ATP) by bacteria and its role in contractility of bladder muscles, such as the detrusor muscle. For this testing, host responses to various urogenital bacteria were modeled through using urothelial bladder cell lines and then with myofibroblast contraction assays. The responses were measured through calcium influx, gene expression, and alpha smooth muscle actin deposition assays.

### Ca²⁺ Influx of Uroepithelial Cell by Bacterial Supernatants

Intracellular calcium has many roles inside the cell and regulates important mechanisms such as gene expression, metabolism, and proliferation. This influx can be rapidly induced in the presence of ATP and has been previously shown to be produced extracellularly by some bacteria in vitro. In patients with urinary infections, antibiotics are often administered. This reduces the number of bacteria in the lumen where they are exposed to therapeutic concentrations of the antibiotic. However, bacteria can also be embedded intracellularly in the urothelial cells, where only sub-therapeutic concentration of antibiotics may reach.

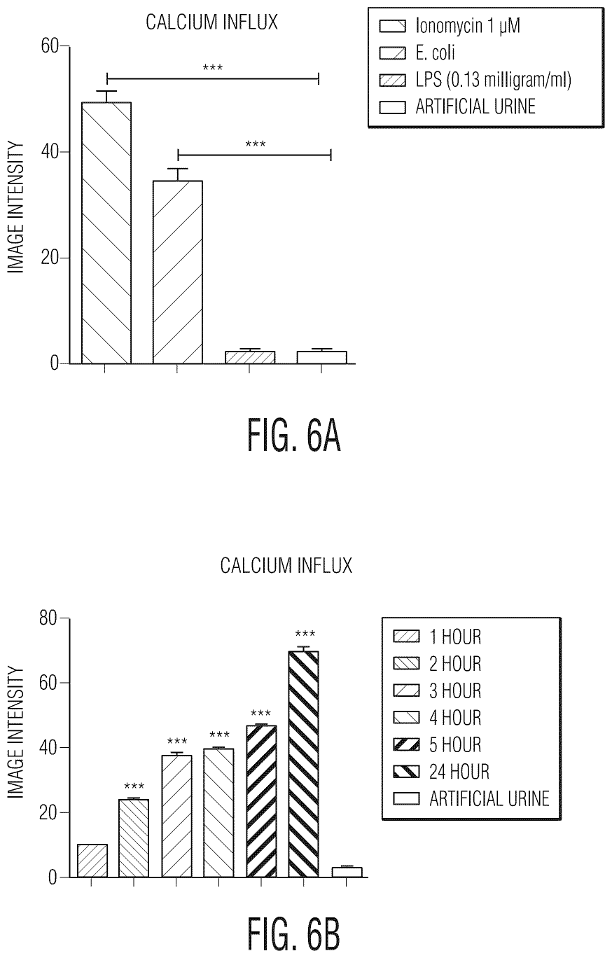

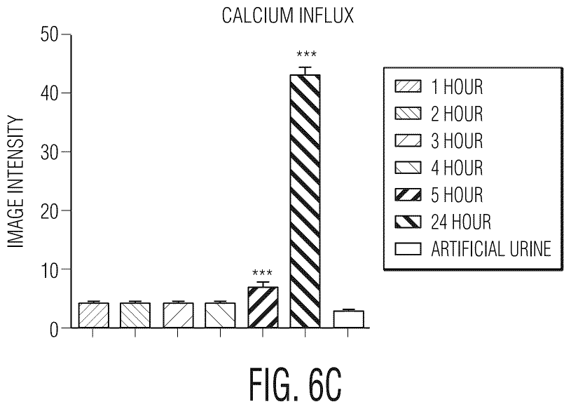

Turning to FIG. 6A, the supernatant of E. coli 1A2 was able to induce the influx of Ca²⁺. Unlike previous reports, testing with lipopolysaccharides (LPS) did not rapidly stimulate the influx of calcium in the model testing. As depicted in FIG. 6B, supernatants from E. coli increased the levels of Ca²⁺ influx before leveling out around the four-hour time point. As depicted in FIG. 6C, the supernatant from E. faecalis 33186 did not demonstrate a significant increase in levels of Ca²⁺ influx until about five hours. In GIS. In FIGS. 6A-6C, each bar represents the total average image intensity 60 seconds after treatment of a duplicate sample. Statistical significance was determined using Tukey's test, p≤0.05, and is designated with three asterisks.

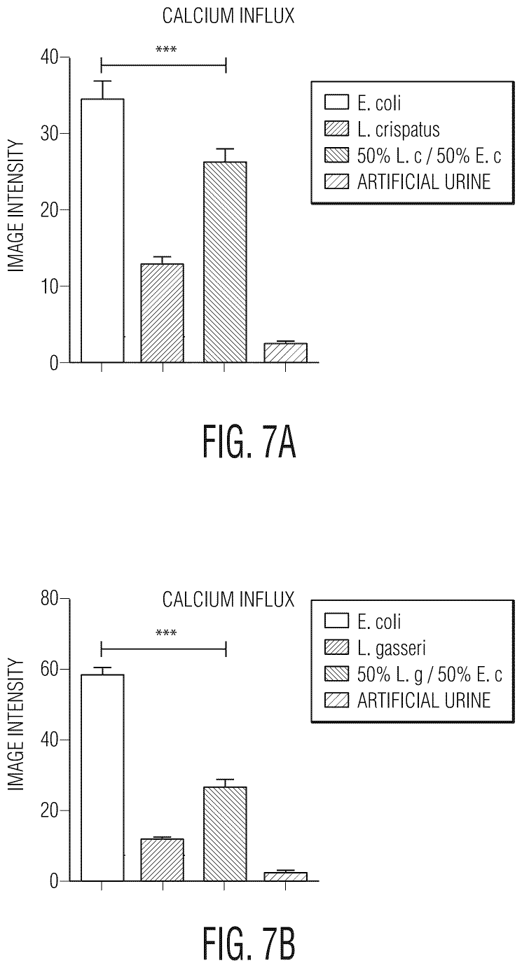

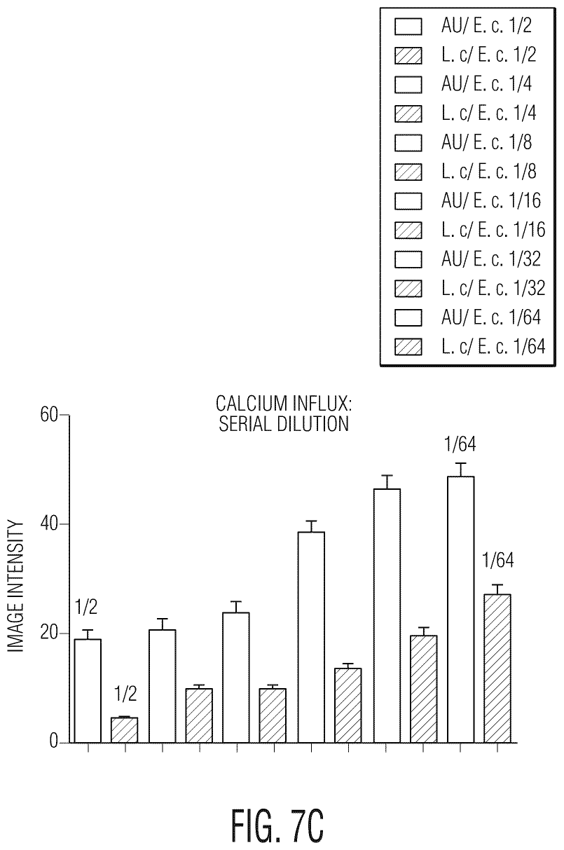

Additional testing calcium influx testing was also completed with supernatants from Lactobacillus crispatus ATCC 33820 and Lactobacillus gasseri strain KE-1 (ATCC Designation No. PTA-125162). As demonstrated in FIGS. 7A-7C, the addition of L. crispatus and L. gasseri supernatants demonstrated a decrease of the calcium influx caused by E. coli supernatants by up to fifty percent. In FIGS. 7A-7C, each point represents the average image intensity of a timepoint. Statistical significance was determined using Tukey's test, p≤0.05, and is designated with three asterisks.

### Quantification of Bacterial Extracellular ATP

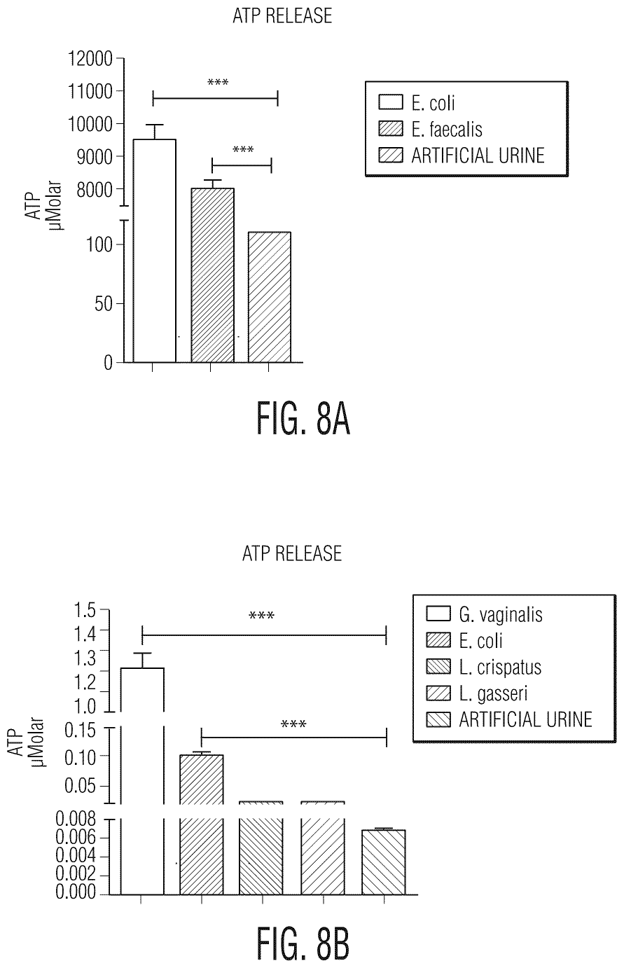

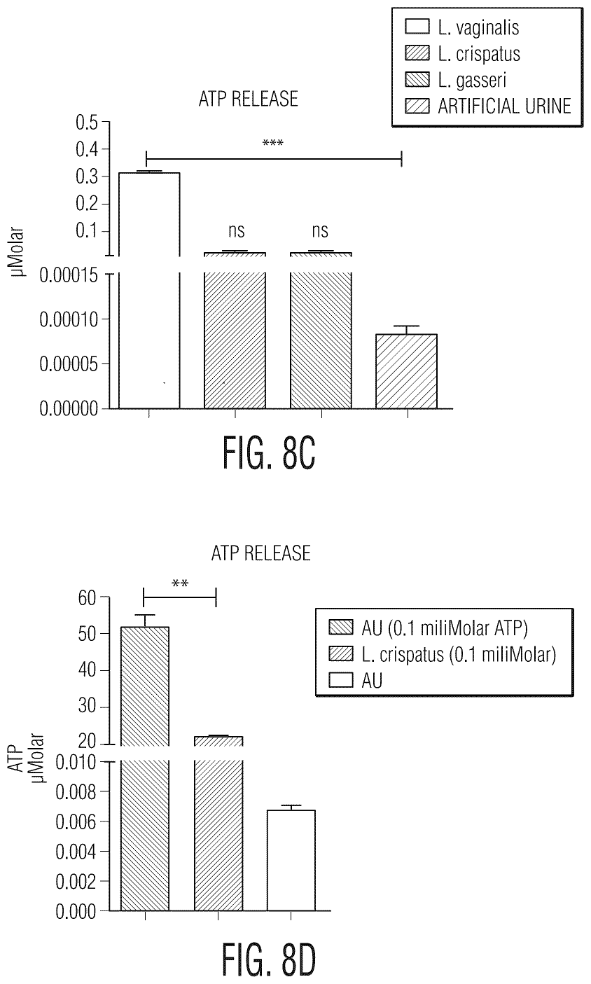

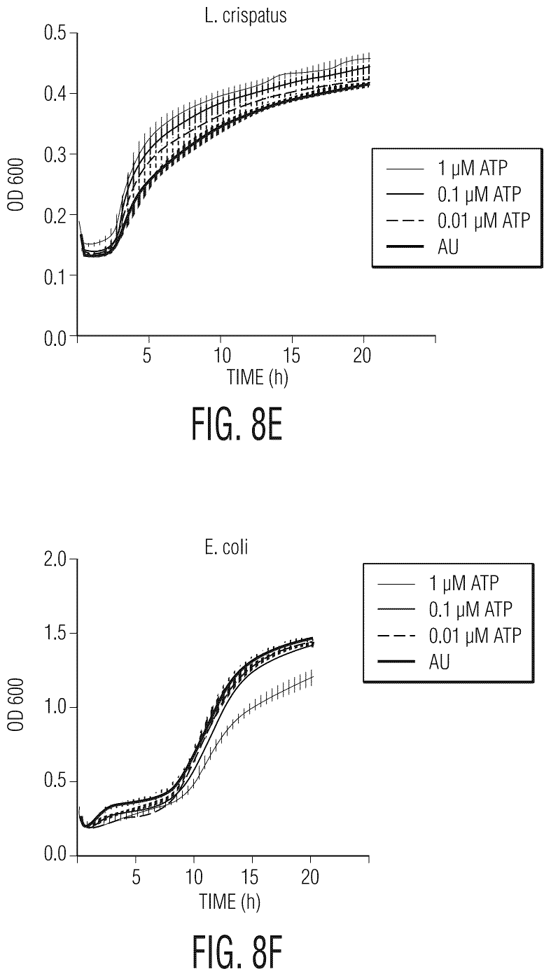

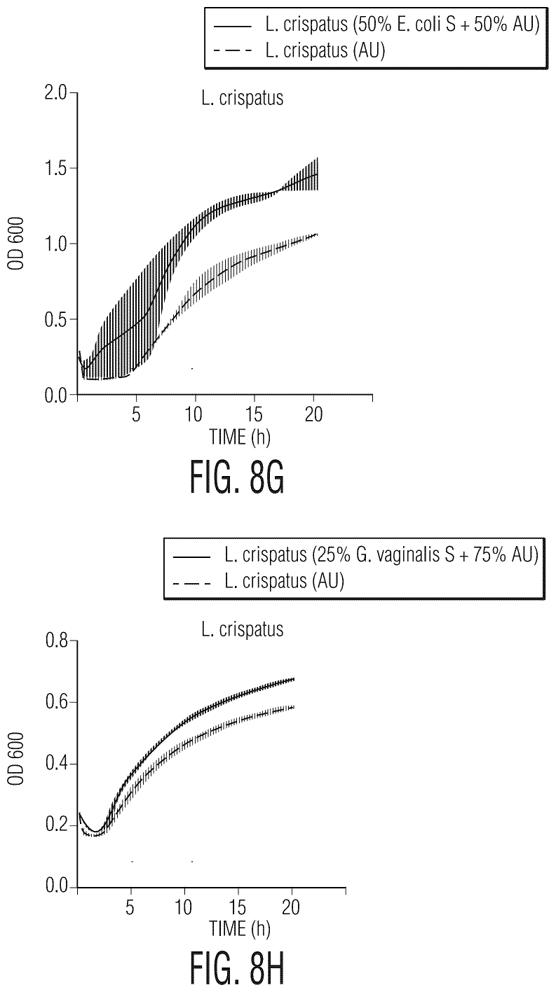

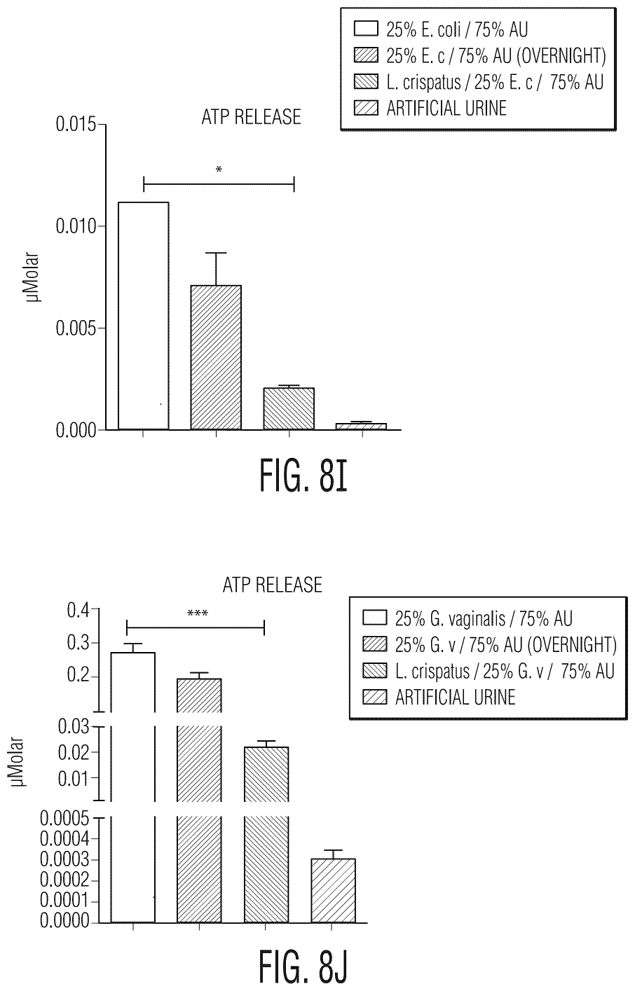

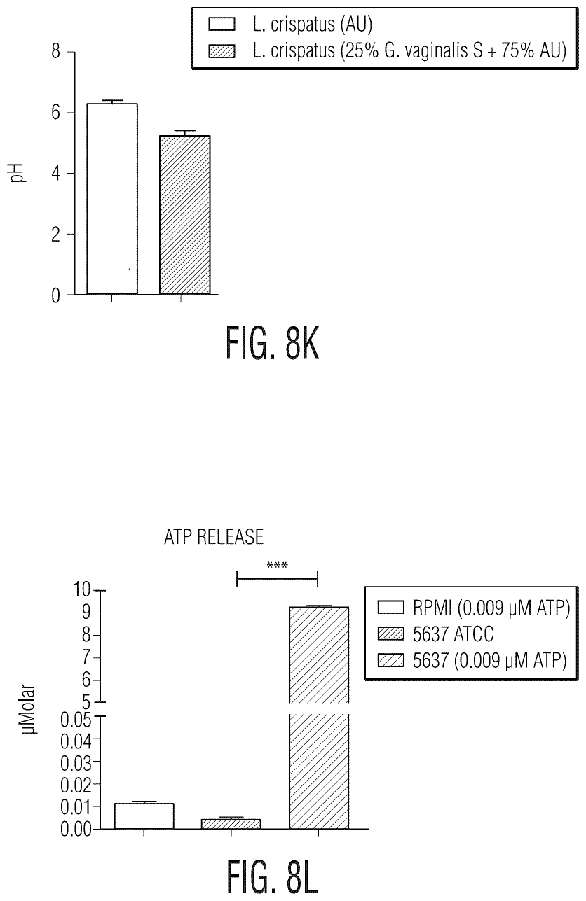

FIGS. 8A-8D, 8I, 8J, and 8L depict a quantification of the amount of extracellular ATP released by various bacterial samples in comparison to controls of AU without bacteria. A luminescent assay was used to quantify the amount of extracellular ATP released by bacterial supernatants. As shown in FIG. 8A, the E. coli and E. faecalis supernatants from overnight cultures contained 10000±900 nM and 8333±557 nM ATP, respectively, which was higher than the control of artificial urine (AU) which contained 111±10 nM. Thus, uropathogenic E. coli can release ATP into AU and cause the influx of calcium. As depicted in FIG. 8B, the E. coli, L. crispatus and L. gasseri supernatants from the overnight culture contained 0.11±0.01 uM 0.025±0.001 uM and 0.023 uM ATP, respectively, which was higher than AU that contained 0.0071±0.0002 uM. In addition, supernatants of urinary microbiota constituents, G. vaginalis ATCC 14018 and L. vaginalis NCFB 2810 contained 1.36±0.24 uM and 0.33±0.032 uM ATP, respectively, which was greater than the AU control of 0.000071±0.000029 uM, as depicted in FIGS. 8B and 8C.

As depicted in FIG. 8D, the amount of ATP remaining when L. crispatus was grown in AU supplemented with 0.1 mM ATP for 24 hours was 22.89±0.98 uM, which was less than half the control (49.2±6.3 uM) (P≤0.0005). To further investigate ATP reduction, L. crispatus was cultured in AU supplemented with different concentrations of ATP (FIG. 8E), as well as in AU supplemented with 50% E. coli supernatant (FIG. 8G), and 25% G. vaginalis supernatant (FIGS. 8H and 8J), as potential natural sources of ATP. The growth of L. crispatus was increased by increasing ATP concentration (FIG. 8E), including that emanating from the E. coli and G. vaginalis supernatants (FIGS. 8H and 8J). Lactobacillus crispatus also reduced the amount of ATP after overnight culture in AU supplemented with 25% of E. coli supernatant (FIG. 8I), and 25% of G. vaginalis supernatant individually (FIG. 8J). As depicted in FIG. 8F, supplementing E. coli with ATP had a somewhat inhibitory effect on its growth. In the presence of ATP or supernatant from G. vaginalis, the pH of L. crispatus became further reduced, as depicted in FIG. 8K. Lastly, as depicted in FIG. 8L, testing of urothelial cells (5637 ATCC) was shown to contain 0.0047±0.0008 uM ATP, and after treatment with 0.009 uM ATP for 2 minutes, the ATP released by the urothelial cells increased to 9.3±0.07 uM. In FIGS. 8A-8L, statistical significance was determined using Tukey's test, p≤0.05, and is designated with three asterisks (p less than 0.05*, 0.01**, 0.001***).

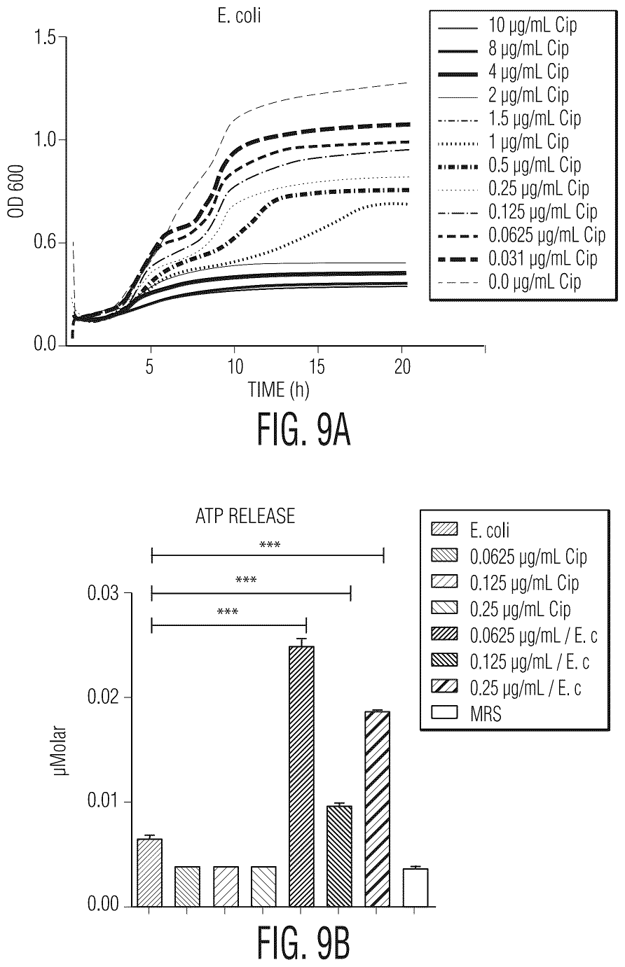

Further testing was completed on E. coli and its release of ATP. Testing was conducted to determine the minimum inhibitory concentration (MIC) and subtherapeutic concentrations of exposure to the antibiotic ciprofloxacin (Cip). After culturing E. coli bacteria with different concentrations of Cip from 10 ug/mL to 0.031 ug/mL, the MIC of the antibiotic against E. coli was 1 to 1.5 ug/mL, as depicted in FIG. 9A. Using MIC concentrations of ciproflaxacin below the MIC of 0.25, 0.125, 0.0625 ug/mL induced E. coli to release more ATP up to 0.0261±0.003 uM, as demonstrated in FIG. 9B (p less than 0.05*, 0.01**, 0.001***).

From the results depicted in FIGS. 8L, 9A, and 9B, it has been shown that subtherapeutic exposure to ciprofloxacin induced E. coli to release more ATP, which in turn could impact urological-neurological conditions and increase bladder contractions. Additionally, the role that the urinary microbiota of incontinence patients might have in uncontrolled voiding was further supported by the finding of an abundant member of the microbiota, G. vaginalis releasing comparatively large amounts of ATP (1.36±0.24 uM) (see FIG. 8B). If these amounts were produced in vivo they would likely cause urothelial cells to release more in the sub-urethral space, potentially leading to mitochondrial dysfunction and cell apoptosis.

### Investigation of the Effect of GABA on Ca²⁺ Influx Caused by ATP and Bacterial Supernatant

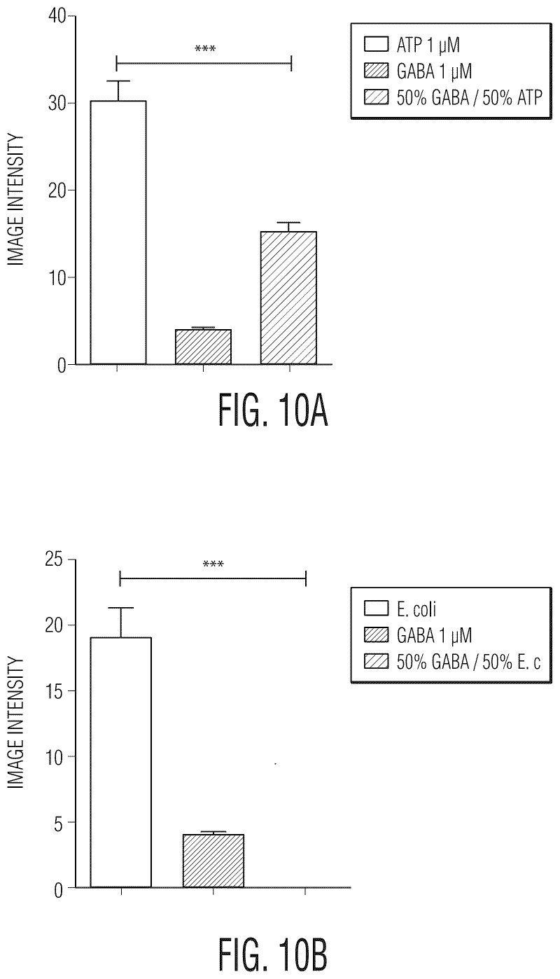

To evaluate the ability of the neurotransmitter γ-aminobutyric acid (GABA) to inhibit the stimulation of calcium influx caused by ATP, AU containing 1 uM GABA was mixed with ATP in AU. As depicted in FIG. 10A, GABA was found to reduce the stimulation of calcium influx caused by ATP. Similarly, to test the ability of GABA to reduce the stimulation of calcium influx caused by bacterial supernatant, GABA was mixed with E. coli supernatant, and as illustrated in FIG. 10B, GABA also demonstrated the ability to inhibit the stimulation of calcium influx caused by E. coli supernatant. Statistical significance was determined using Tukey's test, p≤0.05, and is designated with three asterisks.

Expression of moaA and moaB in Urothelial Cells (5637 ATCC) Exposed to Bacterial Supernatants

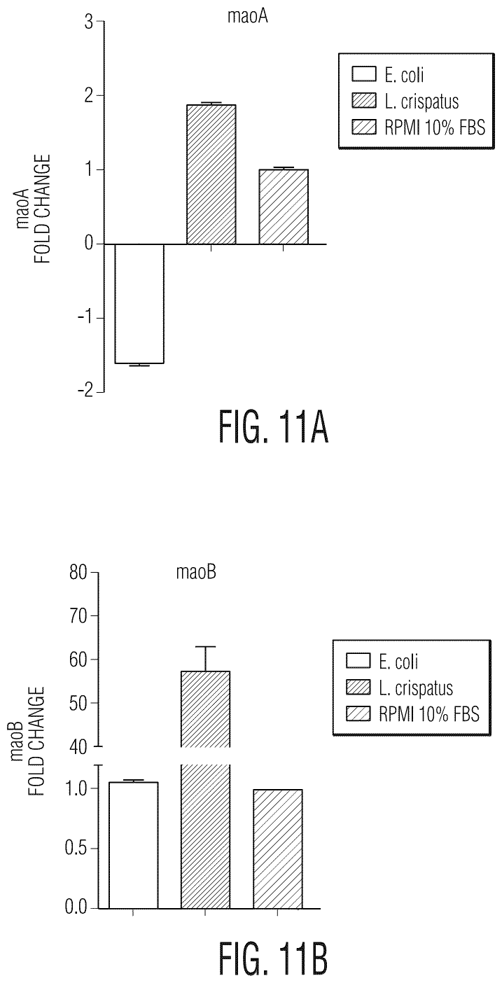

By increasing the level of intracellular calcium, ATP can cause mitochondrial dysfunction. Gene expression for mitochondrial enzymes, moaA and moaB, was measured because of their potential ability to degrade neurotransmitters such as serotonin, norepinephrine, phenylethylamines, and dopamine. As previously mentioned, these neurotransmitters are known to play a role in the frequency of urination, the maximum urine capacity of the bladder, and the contraction of bladder muscles. Supernatants from both pure cultures and mixtures of E. coli and L. crispatus were added to 5637 cell cultures for 3 hours. Expression of genes encoding maoA and moaB were measured by quantitative PCR relative to GAPDH (A and BAs demonstrated in FIG. 11A, the E. coli supernatant caused marginal (1.63±0.05-fold) downregulation in the level of maoA gene expression, whereas, L. crispatus supernatant caused upregulation (1.912±0.093-fold)). The E. coli supernatant demonstrated no effect on maoB gene expression, whereas, L. crispatus supernatant upregulated its expression by 60-fold, as depicted in FIG. 11B.

### Myofibroblast Contraction Assay

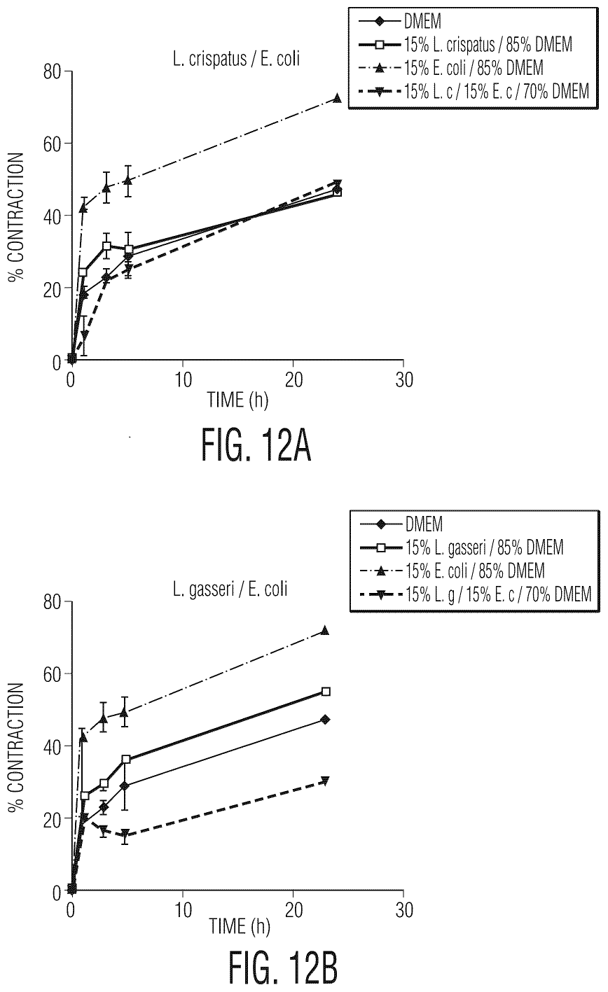

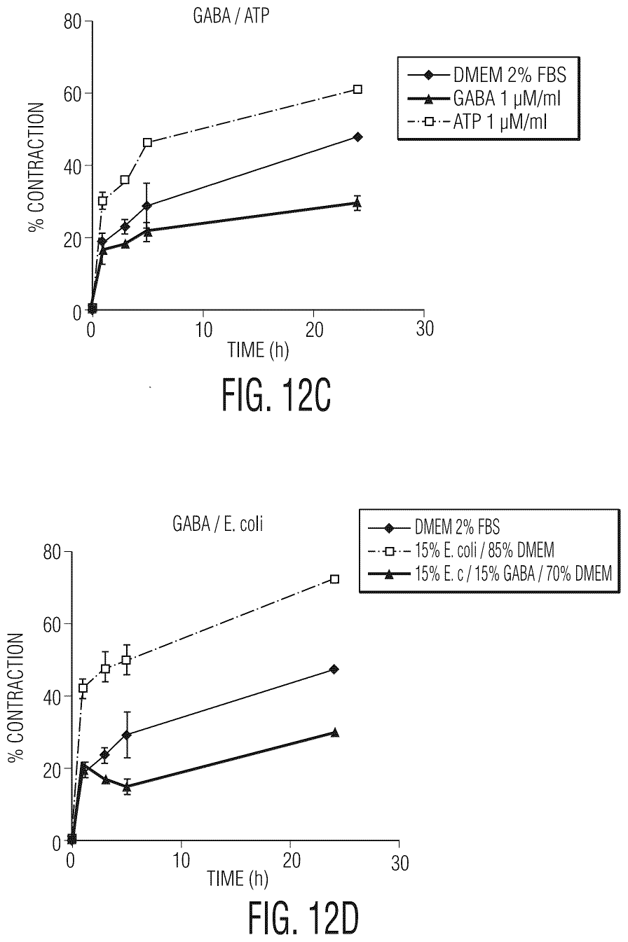

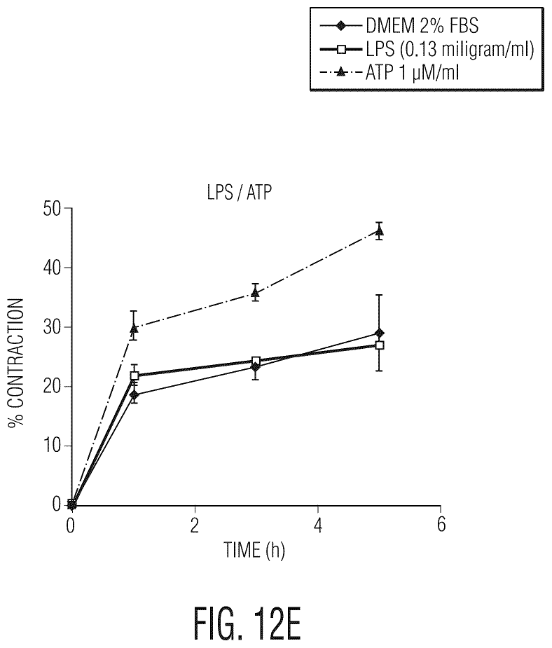

Bacterial supernatants from E. coli, L. crispatus, and L. gasseri strain KE-1 (ATCC Designation No. PTA-125162) were added to a myofibroblast populated collagen matrix, both from pure culture and mixtures with DMEM with 2% FBS. In addition, GABA, ATP and LPS were included as controls. The collagen contraction assay using primary myofibroblast cells seeded on top of a collagen matrix was tested against the bacterial products. As illustrated in FIGS. 12A and 12B, supernatant from cultures of E. coli were able to induce the greatest amount of contraction (72.67%) in the myofibroblast cell line after 24 hours and this reduced when L. crispatus or L. gasseri supernatants were added (48.56%, 29.8%, respectively). As depicted in FIG. 12C, pure ATP caused contraction of myofibroblasts in the first hour (30.37%) and continued for 24 hours (68.56%). While, GABA did not cause contraction in the myofibroblast assay, it inhibited contraction caused by E. coli, as shown in FIG. 12D. Based on previous literature, it is believed that the contraction maybe caused by E. coli was due to LPS. However, as shown in FIG. 12E, after five hours of exposure of LPS to the myofibroblasts, contraction was half that induced by ATP (27.2% versus 57.5%).

### Immunocytochemistry for Intracellular Alpha Smooth Muscle Actin (α-SMA) and Induction of TNF-α by Bacteria

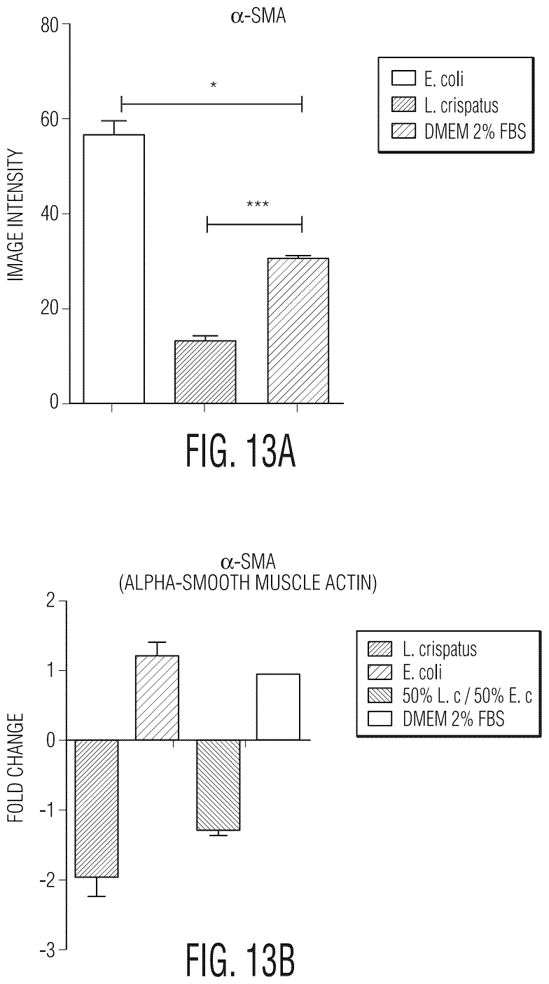

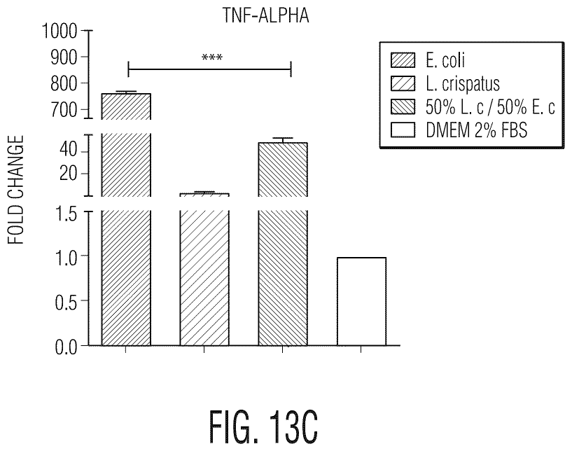

To further confirm myofibroblast contractive abilities in the presence of bacterial compounds, the effect on α-SMA was assessed. Alpha smooth muscle actin (α-SMA) has been suggested to play a role in the production of contractile force during wound healing and fibro-constrictive diseases. Bacterial supernatants from E. coli, L. crispatus, and L. gasseri KE-1 (ATCC Designation No. PTA-125162) and in combination were co-cultured with myofibroblasts for 1 hour. The E. coli supernatant increased the intracellular image intensity (38.3±2.5) which is related to the α-SMA (see FIGS. 13A and 13B) and this was reduced by L. crispatus (8.2±0.3).

Bacteria supernatants, identical to those tested in FIG. 13A, were added to the myofibroblasts that were grown in the collagen matrix and then incubated at 37° C. with 5% CO₂ for 3 hours and tested for gene expression. As shown in FIG. 13B, the E. coli supernatant did not increase the expression of the α-SMA (1.42±0.4-fold change), indicating that the image intensity was just related to the alpha smooth muscle contraction. Lactobacillus crispatus also downregulated the level of α-SMA gene expression (2.245±0.6-fold change) (see FIG. 13B) suggesting it could potentially decrease the amount of α-SMA. In addition, L. crispatus could potentially reduce the expression of α-SMA caused by E. coli. The image intensity was measured by confocal microscopy with the blue-fluorescent DNA stain 4′,6-diamidino-2-phenylindole (DAPI) and Fluorescein isothiocyanate (FITC) dye to show staining of α-SMA. To determine if sustained activation of the calcium channel promoted apoptosis by bacterial components, Tumour Necrosis Factor-alpha (TNF-α) was measured as an indicator. The E. coli caused more than 700-fold upregulation (771±30) of TNF-α (FIG. 13C), whereas, exposure to L. crispatus only resulted in four-fold increase (4.22±1.0). When E. coli and L. crispatus supernatants were mixed and applied to the assay, the expression of TNF-α induced by the E. coli was strongly mitigated (52.47±7.2), as shown in FIG. 13C.

For FIGS. 13A-13C, statistical significance was determined using Tukey's test, p≤0.05, and is designated with three asterisks (p less than 0.05 designated with one asterisk).

From the testing conducted herein, it was supported that L. crispatus and L. gasseri did not release significant amounts of ATP (see FIG. 8B), and L. crispatus was found to be able to reduce ATP levels in AU supplemented with ATP 0.1 mM (see FIG. 8D). In addition, L. crispatus and L. gasseri inhibited the stimulation of calcium influx caused by E. coli-derived compounds, as demonstrated in FIGS. 6A-6C. Preliminary evidence was obtained that some commensal bacteria could degrade or utilize ATP, with L. crispatus reducing its levels in AU. The L. gasseri bacterial strains tested showed ability to up-regulate, or increase, maoA, moaB, and grin-1 expression, enconding enzymes that can degrade biogenic amines neuroactive chemicals (see FIGS. 1A-3B). L. crispatus demonstrated the ability to up-regulate, or increase, maoB (see FIG. 11B), but did not demonstrate the ability to up-regulate maoA or grin-1 gene expression (see FIGS. 1A, 3A, 11A) in a statistically significant fashion. A decrease in the level of these mitochondrial enzymes has been postulated to worsen neurological disorders and may also be another mechanism by which commensal bacteria mitigate the effects of these chemicals.

Lactobacilli are typically restricted to glycolytic and fermentative pathways which produce much less ATP than through the respiratory pathways used by other bacteria. If lactobacilli present in the bladder microbiota or even the vagina, can scavenge ATP it may not only potentially provide an extra energy source for the bacteria, but could sequester it away from the epithelial layer thereby promoting a homeostatic environment. These are important findings, since ATP was shown to cause contraction of myofibroblasts (see FIGS. 12C-12E), suggesting a potential mechanism for premature voiding, and the potential for lactobacilli strains to interfere with this process. However, not all strains of lactobacilli tested were necessarily protective against the effects of ATP. Lactobacillus vaginalis, commonly found in the oral, vaginal, and intestine has been associated with intermediate grades of bacterial vaginosis and was found to release 0.33±0.032 uM ATP (see FIG. 8C), several fold more than E. coli. Not to be bound by theory, but this suggests that certain lactobacilli may in fact be part of the disease process.

The neurotransmitter GABA can be produced by bacteria, including certain species of Lactobacillus, and while it did not cause contraction of myofibroblasts, it could inhibit contraction by E. coli(see FIGS. 12C, 12D). The increase in intracellular calcium levels can result in the secretion of ATP by urothelial cells (see FIG. 8L), with two potential mechanisms likely. ATP may be released via channels, such as the connexin hemichannels, pannexin, as well as several anion channels. It is possible that stimulation of calcium influx in urothelial cells may cause increased expression of vesicular nucleotide transporter (VNUT) in the cell and subsequent release of ATP into the sub-urethral and muscle layer causing bladder contraction. The alternative is for a continuously activated calcium channel leading to mitochondrial calcium overload, apoptosis, and release of ATP from urothelial cells.

As previously noted, alpha smooth muscle actin (α-SMA) has been suggested to play a role in the production of contractile forces during wound healing and fibro-constrictive diseases. The results of confocal and qPCR results herein show a direct correlation between the contraction of α-SMA and uropathogenic bacterial induced contraction of the collagen gel contraction model, as demonstrated in FIG. 13A. There was also a direct correlation between α-SMA relaxation and the down-regulation of α-SMA expression, and relaxation induced by L. crispatus. Increased intracellular calcium levels can drive the urothelial cells to the apoptosis phase. TNF-alpha can be an inducer of apoptosis [22], and so the ability of L. crispatus to reduce the E. coli-stimulated upregulation of this gene in myofibroblast cells, could be significant (see FIG. 13B).

In summary, commensal members of the urinary microbiota, in particular L. crispatus and L. gasseri, can mitigate the ability of uropathogenic E. coli to stimulate pathways associated with conditions such as UUI.

The composition can be in a wide variety of forms such as, for example, simple solutions (water-based or oil-based), solid forms (e.g. gels or sticks), lotions, suspensions, creams, gels milks, salves, ointments, sprays, emulsions, oils resins, aerosols, and the like. The compositions could be in liquid form of various viscosities. Preferably, the compositions useful in the present disclosure are soluble to facilitate their formulation for administration to a user.

### Carrier

The compositions can include a carrier. The carrier can be any dermatologically acceptable carrier. As used herein, “dermatologically acceptable carrier” generally refers to a carrier that is suitable for topical application to a subject and is compatible with compositions including the modification agents of lactobacilli or ferments thereof. Liquid carrier materials suitable for use in the instant disclosure include those well-known for use in the cosmetic, pharmaceutical, and medical arts as a basis for ointments, lotions, creams, salves, aerosols, gels, suspensions, sprays, foams, washes, and the like, and may be used in their established levels. In some embodiments, the carrier can comprise from about 0.01% to about 99.98% (by total weight of the composition), depending on the carrier used.

Preferable carrier materials include polar solvent materials, such as water. Other potential carriers include emollients, humectants, polyols, surfactants, esters, perfluorocarbons, silicones, and other pharmaceutically acceptable carrier materials. In one embodiment, the carrier is volatile, allowing for immediate deposition of the antimicrobial ingredient to the desired surface while improving overall usage experience of the product by reducing drying time. Non-limiting examples of these volatile carriers include 5c5t Dimethicone, Cyclomethicone, Methyl Perfluoroisobutyl Ether, Methyl Perfluorobutyl Ether, Ethyl Perfluoroisobutyl Ether and Ethyl Perfluorobutyl Ether.

Where the composition forms a wetting composition, such as described below for use with a wet wipe, the composition will typically include water. The compositions can suitably comprise water in an amount of from about 0.01% (by total weight of the composition) to about 99.98% (by total weight of the composition), or from about 1.00% (by total weight of the composition) to about 99.98% (by total weight of the composition), or from about 50.00% (by total weight of the composition) to about 99.98% (by total weight of the composition), or from about 75.00% (by total weight of the composition) to about 99.98% (by total weight of the composition). In some embodiments, water can comprise an amount from about 50.00% (by total weight of the composition) to about 70.00% (by total weight of the composition). In some embodiments, water can comprise an amount greater than 90.00% (by total weight of the composition).

### Emollients

In one embodiment, the compositions can optionally include one or more emollients, which typically act to soften, soothe, and otherwise lubricate and/or moisturize the skin. Suitable emollients that can be incorporated into the compositions include oils such as alkyl dimethicones, alkyl methicones, alkyldimethicone copolyols, phenyl silicones, alkyl trimethylsilanes, dimethicone, dimethicone crosspolymers, cyclomethicone, lanolin and its derivatives, fatty esters, fatty acids, glycerol esters and derivatives, propylene glycol esters and derivatives, alkoxylated carboxylic acids, alkoxylated alcohols, fatty alcohols, and combinations thereof.

Some embodiments of the compositions may include one or more emollients in an amount of from about 0.01% (by total weight of the composition) to about 20% (by total weight of the composition), or from about 0.05% (by total weight of the composition) to about 10% (by total weight of the composition), or from about 0.10% (by total weight of the composition) to about 5% (by total weight of the composition).

### Esters

In some embodiments, the compositions include one or more esters. The esters may be selected from cetyl palmitate, stearyl palmitate, cetyl stearate, isopropyl laurate, isopropyl myristate, isopropyl palmitate, and combinations thereof. The fatty alcohols include octyldodecanol, lauryl, myristyl, cetyl, stearyl, behenyl alcohol, and combinations thereof. The fatty acids can include, but are not limited to, capric acid, undecylenic acid, lauric acid, Myristic acid, palmitic acid, stearic acid, oleic acid, linoleic acid, arachidic acid, and behenic acid. Ethers such as eucalyptol, ceteraryl glucoside, dimethyl isosorbic polyglyceryl-3 cetyl ether, polyglyceryl-3 decyltetradecanol, propylene glycol myristyl ether, and combinations thereof can also suitably be used as emollients. Other suitable ester compounds for use in the antimicrobial compositions or the present disclosure are listed in the International Cosmetic Ingredient Dictionary and Handbook, 11th Edition, CTFA, (January, 2006) ISBN-10: 1882621360, ISBN-13: 978-1882621361, and in the 2007 Cosmetic Bench Reference, Allured Pub. Corporation (Jul. 15, 2007) ISBN-10: 1932633278, ISBN-13: 978-1932633276, both of which are incorporated by reference herein to the extent they are consistent herewith.

### Humectants

Humectants that are suitable as carriers in the compositions of the present disclosure include, for example, glycerin, glycerin derivatives, hyaluronic acid, hyaluronic acid derivatives, betaine, betaine derivatives, amino acids, amino acid derivatives, glycosaminoglycans, glycols, polyols, sugars, sugar alcohols, hydrogenated starch hydrolysates, hydroxy acids, hydroxy acid derivatives, salts of PCA and the like, and combinations thereof. Specific examples of suitable humectants include honey, sorbitol, hyaluronic acid, sodium hyaluronate, betaine, lactic acid, citric acid, sodium citrate, glycolic acid, sodium glycolate, sodium lactate, urea, propylene glycol, butylene glycol, pentylene glycol, ethoxydiglycol, methyl gluceth-10, methyl gluceth-20, polyethylene glycols (as listed in the International Cosmetic Ingredient Dictionary and Handbook such as PEG-2 through PEG 10), propanediol, xylitol, maltitol, or combinations thereof.

The compositions of the disclosure may include one or more humectants in an amount of about 0.01% (by total weight of the composition) to about 20% (by total weight of the composition), or about 0.05% (by total weight of the composition) to about 10% by total weight of the composition), or about 0.1% (by total weight of the composition) to about 5.0% (by total weight of the composition).

### Surfactants

In some embodiments, the composition can include one or more surfactants. In an embodiment where the composition is included in a wipe, the composition may also likely include one or more surfactants. These may be selected from anionic, cationic, nonionic, zwitterionic, and amphoteric surfactants. Amounts of surfactants may range from 0.01 to 30%, or from 10 to 30%, or from 0.05 to 20%, or from 0.10 to 15% by total weight of the composition. In some embodiments, such as when the wetting composition is used with a wipe, the surfactant can comprise less than 5% by total weight of the wetting composition.

Suitable anionic surfactants include, but are not limited to, C₈ to C₂₂ alkane sulfates, ether sulfates and sulfonates. Among the suitable sulfonates are primary C₈ to C₂₂ alkane sulfonate, primary C₈ to C₂₂ alkane disulfonate, C₈ to C₂₂ alkene sulfonate, C₈ to C₂₂ hydroxyalkane sulfonate or alkyl glyceryl ether sulfonate. Specific examples of anionic surfactants include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, potassium lauryl sulfate, sodium trideceth sulfate, sodium methyl lauroyl taurate, sodium lauroyl isethionate, sodium laureth sulfosuccinate, sodium lauroyl sulfosuccinate, sodium tridecyl benzene sulfonate, sodium dodecyl benzene sulfonate, sodium lauryl amphoacetate and mixtures thereof. Other anionic surfactants include the C₈ to C₂₂ acyl glycinate salts. Suitable glycinate salts include sodium cocoylglycinate, potassium cocoylglycinate, sodium lauroylglycinate, potassium lauroylglycinate, sodium myristoylglycinate, potassium myristoylglycinate, sodium palmitoylglycinate, potassium palmitoylglycinate, sodium stearoylglycinate, potassium stearoylglycinate, ammonium cocoylglycinate and mixtures thereof. Cationic counter-ions to form the salt of the glycinate may be selected from sodium, potassium, ammonium, alkanolammonium and mixtures of these cations.

Suitable cationic surfactants include, but are not limited to alkyl dimethylamines, alkyl amidopropylamines, alkyl imidazoline derivatives, quaternised amine ethoxylates, and quaternary ammonium compounds.

Suitable nonionic surfactants include, but are not limited to, alcohols, acids, amides or alkyl phenols reacted with alkylene oxides, especially ethylene oxide either alone or with propylene oxide. Specific nonionics are C₆ to C₂₂ alkyl phenols-ethylene oxide condensates, the condensation products of C₈ to C₁₃ aliphatic primary or secondary linear or branched alcohols with ethylene oxide, and products made by condensation of ethylene oxide with the reaction products of propylene oxide and ethylenediamine. Other nonionics include long chain tertiary amine oxides, long chain tertiary phosphine oxides and dialkyl sulphoxides, alkyl polysaccharides, amine oxides, block copolymers, castor oil ethoxylates, ceto-oleyl alcohol ethoxylates, ceto-stearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, natural alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, synthetic alcohol ethoxylates, tall oil fatty acid ethoxylates, tallow amine ethoxylates and trid tridecanol ethoxylates.

Suitable zwitterionic surfactants include, for example, alkyl amine oxides, alkyl hydroxysultaines, silicone amine oxides, and combinations thereof. Specific examples of suitable zwitterionic surfactants include, for example, 4-[N,N-di(2-hydroxyethyl)-N-octadecylammonio]-butane-1-carboxylate, S—[S-3-hydroxypropyl-S-hexadecylsulfonio]-3-hydroxypentane-1-sulfate, 3-[P,P-diethyl-P-3,6,9-trioxatetradexopcylphosphonio]-2-hydroxypropane-1-phosphate, 3-[N,N-dipropyl-N-3-dodecoxy-2-hydroxypropylammonio]-propane-1-phosphonate, 3-(N,N-dimethyl-N-hexadecylammonio)propane-1-sulfonate, 3-(N,N-dimethyl-N-hexadecylammonio)-2-hydroxypropane-1-sulfonate, 4-[N,N-di(2-hydroxyethyl)-N-(2-hydroxydodecyl)ammonio]-butane-1-carboxylate, 3-[S-ethyl-S-(3-dodecoxy-2-hydroxypropyl)sulfonio]-propane-1-phosphate, 3-[P,P-dimethyl-P-dodecylphosphonio]-propane-1-phosphonate, 5-[N,N-di(3-hydroxypropyl)-N-hexadecylammonio]-2-hydroxy-pentane-1-sulfate, lauryl hydroxysultaine and combinations thereof.

Suitable amphoteric surfactants include, but are not limited to, derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Illustrative amnphoterics are coco dimethyl carboxymethyl betaine, cocoamidopropyl betaine, cocobetaine, oleyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, cocoamphoacetates, and combinations thereof. The sulfobetaines may include stearyl dimethyl sulfopropyl betaine, lauryl dimethyl sulfoethyl betaine, lauryl bis-(2-hydroxyethyl) sulfopropyl betaine and combinations thereof.

### Rheology Modifiers

Optionally, one or more rheology modifiers, such as thickeners, may be added to the composition. Suitable rheology modifiers are compatible with the skin microbiota balancing agent. As used herein, “compatible” refers to a compound that, when mixed with the skin microbiota balancing agent, does not adversely affect the properties of the skin microbiota balancing agent.

A thickening system is used in the compositions to adjust the viscosity and stability of the compositions. Specifically, thickening systems prevent the composition from running off of the hands or body during dispensing and use of the composition. When the composition is used with a wipe product, a thicker formulation can be used to prevent the composition from migrating from the wipe substrate.

The thickening system should be compatible with the compounds used in the present disclosure; that is, the thickening system, when used in combination with the skin microbiota balancing agent, should not precipitate out, form a coacervate, or prevent a user from perceiving the conditioning benefit (or other desired benefit) to be gained from the composition. The thickening system may include a thickener which can provide both the thickening effect desired from the thickening system and a conditioning effect to the user's skin.

Thickeners may include, cellulosics, gums, acrylates, starches and various polymers. Suitable examples include but are not limited to hydroxethyl cellulose, xanthan gum, guar gum, potato starch, and corn starch. In some embodiments, PEG-150 stearate, PEG-150 distearate, PEG-175 diisostearate, polyglyceryl-10 behenate/eicosadioate, disteareth-100 IPDI, polyacrylamidomethylpropane sulfonic acid, butylated PVP, and combinations thereof may be suitable.

While the viscosity of the compositions will typically depend on the thickener used and the other components of the compositions, the thickeners of the compositions suitably provide for a composition having a viscosity in the range of greater than 1 cP to about 30,000 cP or more. In another embodiment, the thickeners provide compositions having a viscosity of from about 100 cP to about 20,000 cP. In yet another embodiment, thickeners provide compositions having a viscosity of from about 200 cP to about 15,000 cP. In embodiments where the compositions are included in a wipe, the viscosity may range from about 1 cP to about 2000 cP. In some embodiments, it is preferable to have a viscosity of the composition be less than 500 cP.

When including a thickening system, the compositions of the present disclosure can include the thickening system in an amount of no more than about 20% (by total weight of the composition), or from about 0.01% (by total weight of the composition) to about 20% (by total weight of the composition). In another aspect the thickening system is present in the antimicrobial composition in an amount of from about 0.10% (by total weight of the composition) to about 10% (by total weight of the composition), or from about 0.25% (by total weight of the composition) to about 5% (by total weight of the composition), or from about 0.5% (by total weight of the composition) to about 2% (by total weight of the composition).

In one embodiment, the compositions may include hydrophobic and hydrophilic ingredients, such as a lotion or cream. Generally, these emulsions have a dispersed phase and a continuous phase, and are generally formed with the addition of a surfactant or a combination of surfactants with varying hydrophilic/lipophilic balances (HLB). Suitable emulsifiers include surfactants having HLB values from 0 to 20, or from 2 to 18. Suitable non-limiting examples include Ceteareth-20, Cetearyl Glucoside, Ceteth-10, Ceteth-2, Ceteth-20, Cocamide MEA, Glyceryl Laurate, Glyceryl Stearate, PEG-100 Stearate, Glyceryl Stearate, Glyceryl Stearate SE, Glycol Distearate, Glycol Stearate, Isosteareth-20, Laureth-23, Laureth-4, Lecithin, Methyl Glucose Sesquistearate, Oleth-10, Oleth-2, Oleth-20, PEG-100 Stearate, PEG-20 Almond Glycerides, PEG-20 Methyl Glucose Sesquistearate, PEG-25 Hydrogenated Castor Oil, PEG-30 Dipolyhydroxystearate, PEG-4 Dilaurate, PEG-40 Sorbitan Peroleate, PEG-60 Almond Glycerides, PEG-7 Olivate, PEG-7 Glyceryl Cocoate, PEG-8 Dioleate, PEG-8 Laurate, PEG-8 Oleate, PEG-80 Sorbitan Laurate, Polysorbate 20, Polysorbate 60, Polysorbate 80, Polysorbate 85, Propylene Glycol Isostearate, Sorbitan Isostearate, Sorbitan Laurate, Sorbitan Monostearate, Sorbitan Oleate, Sorbitan Sesquioleate, Sorbitan Stearate, Sorbitan Trioleate, Stearamide MEA, Steareth-100, Steareth-2, Steareth-20, Steareth-21. The compositions can further include surfactants or combinations of surfactants that create liquid crystalline networks or liposomal networks. Suitable non-limiting examples include OLIVEM 1000 (INCI: Cetearyl Olivate (and) Sorbitan Olivate (available from HallStar Company (Chicago, IL)); ARLACEL LC (INCI: Sorbitan Stearate (and) Sorbityl Laurate, commercially available from Croda (Edison, NJ)); CRYSTALCAST MM (INCI: Beta Sitosterol (and) Sucrose Stearate (and) Sucrose Distearate (and) Cetyl Alcohol (and) Stearyl Alcohol, commercially available from MMP Inc. (South Plainfield, NJ)); UNIOX CRISTAL (INCI: Cetearyl Alcohol (and) Polysorbate 60 (and) Cetearyl Glucoside, commercially available from Chemyunion (S\grave{h}o Paulo, Brazil)). Other suitable emulsifiers include lecithin, hydrogenated lecithin, lysolecithin, phosphatidylcholine, phospholipids, and combinations thereof.

### Gelling Agents

In some embodiments in which the composition is in the form of a gel, the disperse phase of the gel may be formed from any of a variety of different gelling agents, including temperature responsive (“thermogelling”) compounds, ion responsive compounds, and so forth. Thermogelling systems, for instance, respond to a change in temperature (e.g., increase in temperature) by changing from a liquid to a gel. Generally speaking, the temperature range of interest is from about 25° C. to about 40° C., in some embodiments from about 35° C. to about 39° C., and in one particular embodiment, at the human body temperature (about 37° C.). In some cases, thermogelling block copolymers, graft copolymers, and/or homopolymers may be employed. For example, polyoxyalkylene block copolymers may be used in some embodiments of the present invention to form a thermo-gelling composition. Suitable thermo-gelling compositions may include, for example, homopolymers, such as poly(N-methyl-N-n-propylacrylamide), poly(N-n-propylacrylamide), poly(N-methyl-N-isopropylacrylamide), poly(N-n-propylmethacrylamide), poly(N-isopropylacrylamide), poly(N,n-diethylacrylamide); poly(N-isopropylmethacrylamide), poly(N-cyclopropylacrylamide), poly(N-ethylmethyacrylamide), poly(N-methyl-N-ethylacrylamide), poly(N-cyclopropylmethacrylamide), and poly(N-ethylacrylamide). Still other examples of suitable thermogelling polymers may include cellulose ether derivatives, such as hydroxypropyl cellulose, methyl cellulose, hydroxypropylmethyl cellulose, and ethylhydroxyethyl cellulose. Moreover thermogelling polymers may be made by preparing copolymers between (among) monomers, or by combining such homopolymers with other water-soluble polymers, such as acrylic monomers (e.g., acrylic or methacrylic acid, acrylate or methacrylate, acrylamide or methacrylamide, and derivatives thereof).

### Ion Responsive Compounds

The compositions of the present invention may also include an ion responsive compound. Such compounds are generally well known in the art, and tend to form a gel in the presence of certain ions or at a certain pH. For instance, one suitable class of ion responsive compounds that may be employed in the present invention is anionic polysaccharides. Anionic polysaccharides may form a three-dimensional polymer network that functions as the disperse phase of the gel. Generally speaking, anionic polysaccharides include polysaccharides having an overall anionic charge, as well as neutral polysaccharides that contain anionic functional groups.

### Antimicrobial Agents

In some embodiments, the composition may include one or more antimicrobial agents to increase shelf life. Some suitable antimicrobial agents that may be used in the present disclosure include traditional antimicrobial agents. As used herein, “traditional antimicrobial agents” means compounds that have been historically recognized by regulatory bodies as providing an antimicrobial effect, such as those listed in the European Union's Annex V list of preservatives allowed in cosmetics products. Traditional antimicrobial agents include, but are not limited to: propionic acid and salts thereof; salicylic acid and salts thereof; sorbic acid and salts thereof; benzoic acid and salts and esters thereof; formaldehyde; paraformaldehyde; o-phenylphenol and salts thereof; zinc pyrithione; inorganic sulfites; hydrogen sulfites; chlorobutanol; benzoic parabens, such as methylparaben, propylparaben, butylparaben, ethylparaben, isopropylparaben, isobutylparaben, benzylparaben, sodium methylparaben and sodium propylparaben; dehydroacetic acid and salts thereof; formic acid and salts thereof; dibromohexamidine isethionate; thimerosal; phenylmercuric salts; undecylenic acid and salts thereof; hexetidine; 5-bromo-5-nitro-1,3-dioxane; 2-bromo-2-nitropropane-1,3,-diol; dichlorobenzyl alcohol; triclocarban; p-chloro-m-cresol; triclosan; chloroxylenol; imidazolidinyl urea; polyaminopropyl biguanide; phenoxyethanol, methenamine; quaternium-15; climbazole; DMDM hydantoin; benzyl alcohol; piroctone olamine; bromochlorophene; o-cymen-5-ol; methylchloroisothiazolinone; methylisothiazolinone; chlorophene; chloroacetamide; chlorhexidine; chlorhexidine diacetate; chlorhexidine digluconate; chlorhexidine dihydrochloride; phenoxyisopropanol; alkyl (C12-C22) trimethyl ammonium bromide and chlorides; dimethyl oxazolidine; diazolidinyl urea; hexamidine; hexamidine diisethionate; glutaral; 7-ethylbicyclooxazolidine; chlorphenesin; sodium hydroxymethylglycinate; silver chloride; benzethonium chloride; benzalkonium chloride; benzalkonium bromide; benzylhemiformal; iodopropynyl butylcarbamate; ethyl lauroyl arginate HCl; citric acid and silver citrate.

Other antimicrobial agents that may be added to the compositions of the present disclosure include non-traditional antimicrobial agents that are known to exhibit antimicrobial effects in addition to their primary functions, but that have not historically been recognized as antimicrobial agents by regulatory bodies (such as on the European Union's Annex V list). Examples of these non-traditional antimicrobial agents include, but are not limited to, hydroxyacetophenone, caprylyl glycol, sodium coco-PG dimonium chloride phosphate, phenylpropanol, lactic acid and salts thereof, caprylhydroxamic acid, levulinic acid and salts thereof, sodium lauroyl lactylate, phenethyl alcohol, sorbitan caprylate, glyceryl caprate, glyceryl caprylate, ethylhexylglycerin, p-anisic acid and salts thereof, gluconolactone, decylene glycol, 1,2-hexanediol, glucose oxidase and lactoperoxidase, leuconostoc/radish root ferment filtrate and glyceryl laurate.

The amount of the antimicrobial agents in the compositions is dependent on the relative amounts of other components present within the composition. For example, in some embodiments, an antimicrobial agent can be present in the compositions in an amount between about 0.001% to about 5% (by total weight of the composition), in some embodiments between about 0.01 to about 3% (by total weight of the composition), and in some embodiments, between about 0.05% to about 1.0% (by total weight of the composition). In some embodiments, the antimicrobial agent can be present in the composition in an amount less than 0.2% (by total weight of the composition). However, in some embodiments, the composition can be substantially free of any antimicrobial agents. Thus, in some embodiments, the composition does not include a traditional antimicrobial agent or a non-traditional antimicrobial agent.

### Adjunct Ingredients

The compositions of the present disclosure may additionally include adjunct ingredients conventionally found in cosmetic, pharmaceutical, medical, household, industrial, or personal care compositions/products in an established fashion and at established levels. For example, the compositions may comprise additional compatible pharmaceutically active and compatible materials for combination therapy, such as antioxidants, anti-parasitic agents, antipruritics, antifungals, antiseptic actives, biological actives, astringents, keratolytic actives, local anaesthetics, anti-stinging agents, anti-reddening agents, skin soothing agents, external analgesics, film formers, skin exfoliating agents, sunscreens, and combinations thereof.

Other suitable additives that may be included in the compositions of the present disclosure include compatible colorants, deodorants, emulsifiers, anti-foaming agents (when foam is not desired), lubricants, skin conditioning agents, skin protectants and skin benefit agents (e.g., aloe vera and tocopheryl acetate), solvents (e.g., water soluble glycol and glycol ethers, glycerin, water soluble polyethylene glycols, water soluble polyethylene glycol ethers, water soluble polypropylene glycols, water soluble polypropylene glycol ethers, dimethylisosorbide), solubilizing agents, suspending agents, builders, (e.g., alkali and alkaline earth metal salts of carbonate, bicarbonate, phosphate, hydrogen phosphate, dihydrogen phosphate, sulfate hydrogen sulfate), wetting agents, pH adjusting ingredients (a suitable pH range of the compositions can be from about 3.5 to about 8), chelators, propellants, dyes and/or pigments, and combinations thereof.

Another component that may be suitable for addition to the compositions is a fragrance. Any compatible fragrance may be used. Typically, the fragrance is present in an amount from about 0% (by weight of the composition) to about 5% (by weight of the composition), and more typically from about 0.01% (by weight of the composition) to about 3% (by weight of the composition). In one desirable embodiment, the fragrance will have a clean, fresh and/or neutral scent to create an appealing delivery vehicle for the end consumer.

Organic sunscreens that may be present in the compositions include ethylhexyl methoxycinnamate, avobenzone, octocrylene, benzophenone-4, phenylbenzimidazole sulfonic acid, homosalate, oxybenzone, benzophenone-3, ethylhexyl salicylate, and mixtures thereof.

As previously noted herein, the compositions of the present disclosure may be applied to a delivery mechanism such as a substrate, which in-turn may be used to deliver and/or apply the prebiotic composition to a user's skin. Suitable substrates include a web, such as a wet laid tissue web or air laid web, gauze, cotton swab, transdermal patch, container or holder. Various webs that the composition can be applied to can provide products in the form of wipes, facial tissue, bath tissue, paper towels, napkins, diapers, diaper pants, feminine hygiene products (tampons, pads), gloves, socks, masks or combinations thereof. In some embodiments, a substrate that the composition is applied to can form part of an absorbent article. For example, the composition could be applied to a substrate that can form at least a portion of a bodyside liner of an absorbent article. Particularly preferred applicators include fibrous webs, including flushable and non-flushable cellulosic webs and nonwoven webs of synthetic fibrous material. Useful webs may be wet laid, air laid, meltblown, or spunbonded. Suitable synthetic fibrous material includes meltblown polyethylene, polypropylene, copolymers of polyethylene and polypropylene, bicomponent fibers including polyethylene or polypropylene, and the like. Useful nonwoven webs may be meltblown, coform, spunbond, airlaid, hydroentangled nonwovens, spunlace, bonded carded webs.

In certain embodiments, particularly those in which the composition is applied to a web, it may be desirable that the formulation provide certain physical attributes, such as having a smooth, lubricious, non-greasy feel; the ability to at least partially transfer from the web to the user's skin; the capability to be retained on the web at about room temperature; or the ability to be compatible with the web manufacturing process. In certain embodiments, it is preferred that at least a portion of the composition is transferred from the tissue to the user's skin in use.

The composition may be applied to a web during formation of the web or after the web has been formed and dried, often referred to as off-line or post-treatment. Suitable methods of applying the composition to a web include methods known in the art such as gravure printing, flexographic printing, spraying, WEKO™, slot die coating, or electrostatic spraying. One particularly preferred method of off-line application is rotogravure printing.

In those instances where the composition is added to the web during formation of the web and prior to drying, it may be preferred to employ an application method that incorporates the composition on the surface of the web. One method of adding the prebiotic to the web surface is by applying the composition during creping of the tissue web. Surprisingly, the composition itself may be used as a creping composition or may be combined with other well-known creping compositions to apply the composition to a tissue web without significantly degrading important web properties such as strength, stiffness or sloughing.

In some embodiments where the composition is applied to a delivery mechanism such as a substrate, the composition can be applied in an add-on amount ranging from about 1% to about 500%, or from about 30% to about 400%, or from about 100% to about 350%. Of course, it is contemplated that the composition may be applied to a delivery substance outside of this range and still be within the scope of this disclosure.

Fibrous webs comprising a composition made according to the present disclosure can be incorporated into multi-ply products. For instance, in one aspect, a fibrous web made according to the present disclosure can be attached to one or more other fibrous webs to form a wiping product having desired characteristics. The other webs laminated to the fibrous web of the present disclosure can be, for instance, a wet-creped web, a calendered web, an embossed web, a through-air dried web, a creped through-air dried web, an uncreped through-air dried web, an airlaid web, and the like, and may or may not comprise any skin microbiota balancing agents.

Processes for producing airlaid non-woven basesheets are described in, for example, published U.S. Pat. App. No. 2006/0008621, herein incorporated by reference to the extent it is consistent herewith.

### MATERIALS AND TEST METHODS

### Bacterial Supernatant Preparation

Escherichia coli 1A2 UPEC was maintained on LB agar (Difco, MD), Lactobacillus gasseri ATCC KE-1 (ATCC Designation No. PTA-125162) (urinary isolate), Lactobacillus crispatus ATCC 33820, Enterococcus faecalis ATCC 33186, were maintained on MRS agar (Difco, MD), Gardnerella vaginalis ATCC 14018, Lactobacillus vaginalis NCFB 2810 were maintained on CBA and Gardnerella Selective Agar. For the studies discussed herein, all strains of bacteria were grown in artificial urine which in preliminary experiments was shown not to stimulate the influx of calcium when in the presence of human cell lines.

Supernatants were collected from cultures grown overnight (24 hours) at 37° C. after reaching stationary phase. Cultures were pelleted by centrifugation at 5000 rpm (Eppendorf Centrifuge 5804 R) for 15 minutes. The supernatant was pH adjusted to 7.0 with 0.1 Molar HCL or NaOH, filter sterilized with 0.22 um sterile syringe filter, and aliquoted and stored at −20 C° until use. In the case of E. coli and E. faecalis, overnight cultures were diluted 1:100 with fresh artificial urine, returned to incubation at 37° C. and sampled at T=1, 2, 3, 4, 5 and 24 hours for testing. For the experiments involving the addition of supernatants from L. crispatus or L. gasseri to that from uropathogens, the urothelial cells were first treated with L. crispatus or L. gasseri supernatant for one minute, then the uropathogenic supernatant was added. In the case of serial dilution, L. crispatus supernatant was diluted for 6-fold to the E. coli supernatant.

For investigation the subtherapeutic concentration of ciprofloxacin, the L. crispatus was grown in deMan, Rogosa, Sharpe media (MRS, Difco, MD). Growth curves for these bacteria were generated using a plate reader (Eon Biotek, VT) at OD600 and 37° C. to determine exponential phase.

### Cell Culture

Bladder epithelial cells (5637-ATCC HTB-9) were maintained in RPMI 1640 (Roswell-Park Memorial Institute media—Thermo Fisher Scientific, MA) supplemented with 10% fetal bovine serum (FBS) (Thermo Fisher Scientific, Ma.) and 2 mM L-glutamine (Thermo Fisher Scientific, MA.) at 37° C. and 5% CO₂. The media was changed every 48 hours or more regularly if the cells were confluent (90%-100%), after washing by 1×PBS and trypsinization by 0.25% Trypsin-EDTA (1×) (Gibco), with the ratio of 1 to 10. Primary myofibroblast cells were extracted from the palmar fascia during surgery from normal tissue. Primary cultures were maintained in DMEM with 10% fetal bovine serum (FBS; Life Technologies, Carlsbad, CA, USA), 1% L-glutamine (Life Technologies) and 1% antibiotic-antimycotic solution (Life Technologies) at 37° C. in 5% CO₂. All primary cell lines were used up to a maximum of four passages, after which they were discarded.

RNA Isolation and qPCR from Cell Lines

RNA was isolated from the samples (200 ng/uL) using the AMBION BY LIFE TECHNOLOGIES PURELINK™ RNA mini kit (Thermo Fisher Scientific, MA), following the manufacturer's instructions. cDNA was made following the instructions on the APPLIED BIOSYSTEMS High Capacity cDNA Reverse Transcription Kit (Thermo-Fisher Scientific, MA) and PCR was conducted using a MASTER CYCLER gradient PCR thermal cycler (Eppendorf, NY). Using GAPDH as the housekeeping gene, qPCR was set up with each sample being run on the plate in triplicate for each of the conditions. A list of the primer sequences used can be found in Table 1. POWER SYBR GREEN PCR MASTER MIX was used (Thermo Fisher Scientific, MA).

TABLE 1
Primer Sequences

| | Primer Pair | | Gene | Gene | |
|---|---|---|---|---|---|
| Primer | ID | Gene Name | Symbol | ID | Exons |
| GAPDH | H_GAPDH_1 | Glyceraldehyde-3-phosphate | GAPDH | 2597 | 9-10 |
| Sigma 1235 | | dehydrogenase | | | |
| maoA | H_MAOA_1 | Monoamine oxidase A | MAOA | 4128 | 3-5 |
| Sigma 1257 | | | | | |
| maoB | H_MAOB_1 | Monoamine oxidase B | MAOB | 4129 | 12-13 |
| Sigma 1257 | | | | | |
| TNF-alpha | H-TNF-alpha | Tumor necrosis factor | TNF-α | 7124 | 3-4 |
| ThermoFisher | | | | | |
| Scientific | | | | | |
| Hs00174128_m1 | | | | | |
| ACTA2 | H-ACTA2 | Actin, alpha 2, smooth | α-SMA | 11475 | 2-3 |
| ThermoFisher | | muscle, aorta | | | |
| Scientific | | | | | |
| Hs00426835_g1 | | | | | |

### Fluorescent Microscopy of Calcium Influx of 5637 Cells

The influx of calcium was measured using the FLUO-4 DIRECT™ Calcium Assay kit (Invitrogen™, CA). Samples and reagents were prepared according to the protocol manual provided. Ninety-six well plates were seeded with 100 μl of 5637 cells at 1×105 cells/mL in supplemented RPMI and allowed to reach confluency, which occurred at about 48-72 hours. Cells were counted by using the INVITROGEN COUNTESS AUTOMATED CELL COUNTER (Thermo Fisher Scientific, MA.) per the manufacturers' instructions. Fifty microliters of cell culture media were removed from the initial 100 μl and 50 μl of FLUO-4 DIRECT™ calcium reagent was added to each well. The plate was incubated at 37° C. for 30 minutes at room temperature while protected from light. Controls included ionomycin (1 uM, Sigma≥98% HPLC), ATP (1 uM, Sigma A1852), GABA (1 uM, Sigma BioXtra≥99%) and LPS (0.13 milligram/mL, Sigma L3755). The effect of treatments was assessed using a NIKON EPIFLUORESCENCE TS2R scope at 10× magnification at 494 nm for excitation and 516 nm for emission for 60 seconds. The image intensity was calculated using ImageJ and is indicative of Ca²⁺ influx into the urothelial cell's cytoplasmic space from either the extracellular environment or intracellular Ca²⁺ stores (referred to as Ca²⁺ influx).

### Quantification of ATP

The release of ATP from bacterial supernatants was measured using an ATP colorimetric/fluorometric assay kit (Biovision Inc, CA). Samples and reagents were prepared according to the protocol provided. The OD numbers were taken by the BIOTEK EON microplate reader. A luminescent assay kit BACTITER-GLO™ Microbial Cell Viability Assay, G8230) was used to quantify the amount of extracellular ATP released by the bacteria into the supernatant and released by the cells into the cell media. The SYNERGY™ H4 Hybrid Multi-Mode Microplate Reader was used to quantify the amount of extracellular ATP.

### Immunocytochemistry

Myofibroblast cells were cultured in a μ-Slide 8 Well (ibidi, 80826) to become fully confluent (90%-100%). Cell cultures were washed with 1×PBS and fixed with 1 ml 4% paraformaldehyde per well for 10 minutes at room temperature, then 1 ml 0.1-0.2% TRITON X-100 in PBS to each slide was added. After blocking the cells for non-specific staining with BACKGROUND SNIPER (Biocare Medical, BS966), they were incubated for 8 to 10 minutes, washed then incubated overnight at 4° C. in 1% BSA in PBS containing Monoclonal Anti-Actin, α-SMA (Sigma, A2547) diluted 1:200. The cells were washed, excess liquid aspirated, and secondary antibody solution was added (1-10 ug/ml) (ALEXA FLUOR 488 Donkey anti-mouse IgG secondary antibody, ThermoFisher, A-21202) in 1% BSA in PBS. The cells were protected from light and incubated for 45 minutes at room temperature. After washing with PBS, samples were incubated with 100 uL DAPI (Diluted 1:10 000 in PBS) for 5 minutes in the dark. Confocal images were obtained with a NIKON ECLIPSE TI2 (×60 objective lens, Nikon, Canada). Fluorescence intensity measurements were obtained from entire cells and analyzed with Image J software. Control specimens were identical to experimental specimens except they were exposed to irrelevant isotype matched antibody.

Myofibroblast Populated Collagen RNA Extraction and qPCR

After incubation and aspiration of media, the collagen matrix was collected in microcentrifuge tubes for high speed centrifugation for 5 minutes and then the supernatant was discarded. An aliquot of 100 uL pre-warmed 0.25 mg/ml collagenase was added to each well and incubated for 15 minutes at 37° C. RNA was isolated from the samples using the DIRECT-ZOL RNA Miniprep Kit (Zymo Research) following the manufacturer's instructions, and Trizol reagent was used to lyse the samples. The RNA concentration was measured using nanodrop. cDNA was made following the instructions on the APPLIED BIOSYSTEMS High Capacity cDNA Reverse Transcription Kit (Thermo-Fisher Scientific, MA) and PCR was conducted using a MASTERCYCLER gradient PCR thermal cycler (Eppendorf, NY). Quantitative PCR was set up with each sample being run on the plate in triplicate for each of the conditions, as described earlier. GAPDH was also used as the housekeeping gene. A list of the primers used can be found in Supplementary Table 1.

### Immunocytochemistry

Myofibroblast cells were cultured in a μ-Slide 8 Well (ibidi, 80826) to become fully confluent (90%-100%). Cell cultures were washed with 1×PBS and fixed with 1 ml 4% paraformaldehyde per well for 10 minutes at room temperature, then 1 ml 0.1-0.2% Triton X-100 in PBS to each slide was added. After blocking the cells for non-specific staining with Background Sniper (Biocare Medical, BS966), they were incubated for 8 to 10 minutes, washed then incubated overnight at 4° C. in 1% BSA in PBS containing Monoclonal Anti-Actin, α-SMA (Sigma, A2547) diluted 1:200. The cells were washed, excess liquid aspirated, and secondary antibody solution was added (1-10 ug/ml) (Alexa Fluor 488 Donkey anti-mouse IgG secondary antibody, ThermoFisher, A-21202) in 1% BSA in PBS. The cells were protected from light and incubated for 45 minutes at room temperature. After washing with PBS, samples were incubated with 100 uL DAPI (Diluted 1:10 000 in PBS) for 5 minutes in the dark. Confocal images were obtained with a Nikon Eclipse Ti2 (×60 objective lens, Nikon, Canada). Fluorescence intensity measurements were obtained from entire cells and analyzed with Image J software. Control specimens were identical to experimental specimens except they were exposed to irrelevant isotype matched antibody.

Myofibroblast Populated Collagen RNA Extraction and qPCR

After incubation and aspiration of media, the collagen matrix was collected in microcentrifuge tubes for high speed centrifugation for 5 minutes and then the supernatant was discarded. An aliquot of 100 uL pre-warmed 0.25 mg/ml collagenase was added to each well and incubated for 15 minutes at 37° C. RNA was isolated from the samples using the Direct-zol RNA Miniprep Kit (Zymo Research) following the manufacturer's instructions, and Trizol reagent was used to lyse the samples. The RNA concentration was measured using nanodrop. cDNA was made following the instructions on the Applied Biosystems High Capacity cDNA Reverse Transcription Kit (Thermo-Fisher Scientific, MA) and PCR was conducted using a MasterCycler gradient PCR thermal cycler (Eppendorf, NY). Quantitative PCR was set up with each sample being run on the plate in triplicate for each of the conditions, as described earlier. GAPDH was also used as the housekeeping gene. A list of the primers used can be found in Supplementary Table 1.

### Artificial Urine

The artificial urine (AU) used herein was from the formulation from CRC, Critical Reviews in Microbiology, Volume 16, Issue 1, 1988, by McLean, Robert J. C., et a, and is shown in Table 2.

TABLE 2
Artifical Urine formulation

| Component | Concentration (g/L) |
|---|---|
| CaCl₂ · H₂O | 0.651 |
| MgCl₂ · 6H₂O | 0.651 |
| NaCl | 4.6 |
| Na₂SO₄ | 2.3 |
| Sodium citrate | 0.65 |
| Sodium oxalate | 0.02 |
| KH₂PO₄ | 2.8 |
| KCl | 1.6 |
| NH₄Cl | 1.0 |
| Urea | 25.0 |
| Creatine | 1.1 |
| Tryptic soy broth | 10 |

In forming the artificial urine, pH is adjusted to 5.8. If desired, this mixture can be sterilized by filtration with a 0.45- or 0.2 μm membrane filter. The tryptic soy broth is generally added to stimulate bacterial growth.

### Statistics

The data are expressed as mean±SEM. Statistical significance was assessed using one-way ANOVA followed by Tukey's test (GraphPad Prism 5).

### EMBODIMENTS

In view of the foregoing description and examples, the present disclosure provides the following embodiments.

Embodiment 1: A method of preventing or treating incontinence, overactive bladder, or menstrual cramping in a subject, the method comprising: providing a composition, the composition comprising a modification agent comprising a strain of Lactobacillus or a ferment or a metabolite thereof, wherein the modification agent provides neuromodulation activity by up-regulating at least one of maoA enzymes, maoB enzymes, and grin-1 genes; and administering the composition to the subject to prevent or treat incontinence, overactive bladder, or menstrual cramping.

Embodiment 2: The method of embodiment 1, wherein the modification agent decreases a level of extracellular ATP in a target environment.

Embodiment 3: The method of embodiment 1 or 2, wherein the modification agent comprises a strain of Lactobacillus gasseri or a ferment or metabolite thereof.

Embodiment 4: The method of embodiment 3, wherein the strain of Lactobacillus gasseri or the ferment or metabolite thereof is selected from the group consisting of: Lactobacillus gasseri ATCC Designation No. PTA-125162, Lactobacillus gasseri ATCC Designation No. PTA-125163, and combinations thereof.

Embodiment 5: The method of embodiment 3, wherein the strain of Lactobacillus gasseri or the ferment or metabolite thereof is Lactobacillus gasseri ATCC Designation No. PTA-125162.

Embodiment 6: The method of embodiment 3, wherein the strain of Lactobacillus gasseri or the ferment or metabolite thereof is Lactobacillus gasseri ATCC Designation No. PTA-125163.

Embodiment 7: The method of any one of the preceding embodiments, further comprising: applying the composition to a substrate.

Embodiment 8: The method of embodiment 7, wherein the substrate forms at least a portion of an absorbent article.

Embodiment 9: The method of embodiment 7, wherein the substrate forms at least a portion of a wipe.

Embodiment 10: The method of any one of embodiments 1-6, wherein the composition is in the form of a liquid to be administered to the subject by topical application.

Embodiment 11: The method of any one of embodiments 1-6, wherein the composition is in the form of a pill configured to be administered to the subject by ingestion.

Embodiment 12: A method of preventing or treating incontinence, overactive bladder, or menstrual cramping in a subject, the method comprising: providing a composition, the composition comprising a modification agent comprising a strain of Lactobacillus or a ferment or a metabolite thereof, wherein the modification agent decreases an amount of ATP in a target environment; and administering the composition to the subject to prevent or treat incontinence, overactive bladder, or menstrual cramping.

Embodiment 13: The method of embodiment 12, wherein the modification agent comprises a strain of Lactobacillus crispatus or a ferment or a metabolite thereof.

Embodiment 14: A composition comprising: a carrier; and a modification agent comprising a strain of Lactobacillus gasseri or a ferment or a metabolite thereof selected from the group consisting of: Lactobacillus gasseri ATCC Designation No. PTA-125162, Lactobacillus gasseri ATCC Designation No. PTA-125163, and combinations thereof.

Embodiment 15: The composition of embodiment 14, wherein the strain of Lactobacillus gasseri or the ferment or metabolite thereof is Lactobacillus gasseri ATCC Designation No. PTA-125162.

Embodiment 16: The composition of embodiment 14, wherein the strain of Lactobacillus gasseri or the ferment or metabolite thereof is Lactobacillus gasseri ATCC Designation No. PTA-125163.

Embodiment 17: The composition of any one of embodiments 14-16, wherein the modification agent provides neuromodulation activity by up-regulating at least one of maoA enzymes, maoB enzymes, and grin-1 genes.

Embodiment 18: The composition of any one of embodiments 14-17, wherein the composition is applied to a substrate.

Embodiment 19: The composition of any one of embodiments 14-17, wherein the composition is in the form of a liquid.

Embodiment 20: The composition of any one of embodiments 14-17, wherein the composition is in the form of a pill.

1. A method of preventing or treating incontinence or overactive bladder in a subject, the method comprising:
providing a composition, the composition comprising a modification agent comprising a strain of Lactobacillus or a ferment or a metabolite thereof, wherein the modification agent provides neuromodulation activity by up-regulating at least one of maoA enzymes, maoB enzymes, and grin-1 genes; and
administering the composition to the subject to prevent or treat incontinence or overactive bladder.

2. The method of claim 1, wherein the modification agent decreases a level of extracellular ATP in a target environment.

3. The method of claim 1, wherein the modification agent comprises a strain of Lactobacillus gasseri or a ferment or metabolite thereof.

4. The method of claim 3, wherein the strain of Lactobacillus gasseri or the ferment or metabolite thereof is selected from the group consisting of: Lactobacillus gasseri ATCC Designation No. PTA-125162, Lactobacillus gasseri ATCC Designation No. PTA-125163, and combinations thereof.

5. The method of claim 1, wherein the strain of Lactobacillus gasseri or the ferment or metabolite thereof is Lactobacillus gasseri ATCC Designation No. PTA-125162.

6. The method of claim 1, wherein the strain of Lactobacillus gasseri or the ferment or metabolite thereof is Lactobacillus gasseri ATCC Designation No. PTA-125163.

7. The method of claim 1, further comprising:
applying the composition to a substrate.

8. The method of claim 7, wherein the substrate forms at least a portion of an absorbent article.

9. The method of claim 7, wherein the substrate forms at least a portion of a wipe.

10. The method of claim 1, wherein the composition is in the form of a liquid to be administered to the subject by topical application.

11. The method of claim 1, wherein the composition is in the form of a pill configured to be administered to the subject by ingestion.

12. A method of preventing or treating incontinence or overactive bladder in a subject, the method comprising:
providing a composition, the composition comprising a modification agent comprising a strain of Lactobacillus or a ferment or a metabolite thereof, wherein the modification agent decreases an amount of ATP in a target environment; and
administering the composition to the subject to prevent or treat incontinence or overactive bladder.

13. The method of claim 12, wherein the modification agent comprises a strain of Lactobacillus crispatus or a ferment or metabolite thereof.

14.-20. (canceled)
